(19) United States
(12) Patent Application Publication
(10) Pub. No.: US 2011/0011740 A1
(43) Pub. Date: Jan. 20, 2011

(54) CAPILLARY IMMUNOASSAY SYSTEMS AND METHODS

(76) Inventors: David J. Roach, Los Gatos, CA (US); Tom W. Yang, Cupertino, CA (US)

Correspondence Address: COOLEY LLP;ATTN: Patent Group, Suite 1100, 777 - 6th Street, NW, WASHINGTON, DC, 20001, US

(21) Appl. No.: 12/762,830

(22) Filed: Apr. 19, 2010

Related U.S. Application Data
(60) Provisional application No. 61/170,562, filed on Apr. 17, 2009.

(51) Int. Cl. G01N 27/26; C07K 1/26

(52) U.S. Cl. 204/452; 204/603

(57) ABSTRACT

An automated assay system is described with stations for placement of materials to be used in an assay of materials inside capillaries and an automated gripper for manipulating capillaries. The system includes a separation/detection station where reactions inside the capillaries take place and photoemissions from the capillary reactions are detected. The photoemissions from the capillaries may be displayed as line graphs or in columns of a pseudo-gel image resembling the familiar Western gel blot. An automated control system has a user interface by which an operator can select a run protocol and define the locations of samples and reagents to be used in the protocol run. Following the setup the control system will cause the automated system to execute the protocol, then display the results in a selected display format.

### CROSS-REFERENCE TO RELATED APPLICATION

This application claims priority to and the benefit of U.S. Provisional Patent Application Ser. No. 61/170,562, filed Apr. 17, 2009, and entitled “Capillary Immunoassay Systems and Methods,” which is incorporated herein by reference in its entirety.

### FIELD OF THE INVENTION

Embodiments of this invention generally relate to compositions, methods and systems for detecting biological substances. More particularly, embodiments of the present invention relates to a capillary immunoassay system and method.

### BACKGROUND OF THE INVENTION

A number of methods and systems have been developed for conducting various processing and/or analyses of biological substances, such as those described in U.S. Pat. No. 6,423,536 for temperature cycling processes, U.S. Pat. Nos. 5,843,680, 5,784,154, 5,395,502, and 5,137,609 for separation assay methods, U.S. Pat. No. 5,785,926 for a capillary transport system, international publication WO 94/13829 for an isoelectric focusing separation assay system, and U.S. Pat. No. 6,430,512 for a chromatographic fluorescence separation and display system.

More recently, U.S. Pat. App. Pub. Nos. 20060029978 and 20030032035, the disclosures of all of which are incorporated herein by reference in their entireties, describe apparatus and methods for assaying microliter volumes of cellular material by separating constituent substances of the material in a fluid chamber such as a capillary, binding the separated substances in place, then eliciting an optical response from the bound substances such as fluorescence or chemiluminescence. The resulting information has content similar to that of a Western gel blot but without the complex, extensive and time-consuming handling and processing steps that adversely affect reproducibility and make automation difficult. This technique also has advantages such as the ability to assay very small volumes of materials such as those on the cellular level, and good sensitivity due to the ability to receive optical data from chemiluminescence for as long as necessary to obtain a desirable output signal level. However, it would be desirable to automate this technique so that multiple samples may be analyzed simultaneously or in rapid succession with ease and robustness while only consuming minimal volumes of precious reagents and expensive disposables. Thus, further developments are needed.

### SUMMARY OF THE INVENTION

According to some embodiments of the present invention, an automated assay system is described with stations for placement of materials to be used in an assay of materials inside capillaries and an automated gripper for manipulating capillaries. The system includes a separation/detection station where reactions inside the capillaries take place and photoemissions from the capillary reactions are detected. The photoemissions from the capillaries may be displayed as line graphs or in columns of a pseudo-gel image resembling a Western gel blot. An automated control system has a user interface by which an operator can select a run protocol and define the locations of samples and reagents to be used in the protocol run. Following the setup the control system will cause the automated system to execute the protocol, then display the results in a selected display format.

In one aspect, the present invention provides an automated assay system comprising a separation/detection station, the separation/detection station comprising a first location for conducting capillary electrophoresis and the detection of a fluorescence during and after the electrophoresis.

In various embodiments, the first location comprises a capillary holder comprising: first and second fluid reservoirs; a plurality of recesses which retain a plurality of capillaries in position in the holder with the two ends of the capillaries located at the first and second fluid reservoirs; and electrodes in contact with each of the first and second fluid reservoirs, wherein fluids in the reservoirs are retained at the respective ends of the capillaries by surface tension.

In various embodiments, the separation/detection station comprises a UV lamp, wherein the UV lamp is capable of moving to a position over said first location to shed UV light on the capillaries held by the capillary holder. In some embodiments, the UV lamp is grid lamp. In some embodiments, the UV lamp is low-pressure mercury lamp that generates light at a first wavelength of about 254 nm. In some embodiments, the UV lamp further comprises a phosphor coating to convert the first wavelength of about 254 nm to a second wavelength of about 295 nm.

In various embodiments, the automated assay system further comprises an optics module. The optics module comprises a camera having a lens, the lens is optically connected to the separation/detection station for imaging at the first location. In some embodiments, the camera is a digital camera for imaging both during and after the electrophoresis.

In another aspect, the present invention provides a method of measuring one or more analytes in a sample. Embodiments of the methods comprise: adding an internal standard to the sample, the internal standard comprising a bright standard and a dim standard; loading the sample into a microfluidic device; separating the standard and the analyte by electrophoresis; immobilizing the internal standard and the analyte; capturing a first image comprising the signals generated by the bright standard and the dim standard; detecting the analyte with an antibody to produce a second image; capturing a third image comprising the signal generated by the bright standard; and measuring the analyte by comparing the first image, the second image and the third image.

In various embodiments, the signal generated by the bright standard is brighter than the signal generated by the dim standard. In some embodiments, during electrophoresis the bright standard migrates such that it is located apart from the analyte. In some embodiments, the dim standard migrates such that it is located close to the analyte after the electrophoresis. In some embodiments, the amount of the bright standard is about 5-20 times or more than the dim standard. In an exemplary embodiment, the amount of the bright standard is about 10 times of the dim standard. In some embodiments, the electrophoresis is isoelectric focusing (IEF). In some embodiments, the IEF is performed in a capillary. In some embodiments, the electrophoresis separates the analyte by size, preferably in a capillary.

In yet another aspect, the present invention provides a kit for measuring at least one analyte in a sample using electrophoresis. Embodiments of the kit comprise: an internal standard comprising a bright standard and a dim standard, wherein the bright standard is excessive in comparison to the dim standard so as the to generate a brighter signal when detected, and wherein the bright standard locates apart from the analyte after electrophoresis, and the dim standard located close to the analyte after electrophoresis. In some embodiments, the signal generated by the bright standard is at least twice as bright as the signal generated by the dim standard.

In accordance with the principles of the present invention, in some embodiments, a capillary container is provided in which the capillaries can be shipped from the manufacturer and stored by the user prior to use in an automated micro-volume assay system. The container includes a cover which protects coated capillaries from environmental hazards prior to use. The container holds the capillaries in a vertical position so that the base of the container can be used as a capillary rack in the automated assay system. To enable the container and capillaries to be used in an automated assay system without machine vision, the capillaries are positioned on predetermined center-to-center spacings which can be programmed into the control computer of the assay system.

In various embodiments, the capillary container holds a plurality of capillaries in a vertical position which is suitable for use in an automated assay system and the container comprises: a removable cover which can be secured on top of the container, the cover providing clearance for the upper portions of a plurality of capillaries which are loaded into the container; and a base having a plurality of holes arranged in a grid-like pattern which hold capillaries in an upright vertical position, the holes holding the capillaries in a vertical orientation by circumferentially surrounding the capillaries, the base having a lower portion with positions aligned vertically with the tops of the holes which support the capillaries at the bottom ends of the capillaries.

In some embodiments, the holes are funnel-shaped at the top to provide for ease in insertion of the capillaries into the holes. In some embodiments, the holes are arranged in a grid-like pattern of ninety-six holes of eight rows of twelve holes, whereby a fully loaded container contains ninety-six capillaries. In some embodiments, the holes are arranged in a grid-like pattern of 384 holes of sixteen rows of twenty-four holes, whereby a fully loaded container contains 384 capillaries. In some embodiments, the positions supporting the capillaries at the bottom ends comprise tapered apertures each of which guides an inserted capillary to a position vertically aligned with the top a hole which is slightly larger than the diameter of a capillary. In some embodiments, the base measures approximately 1.6 inches by 2.5 inches. In some embodiments, the grid-like pattern of holes further comprises a plurality of holes with a 0.18 inch center-to-center spacing. In some embodiments, the base exhibits a space between the part of the holes which circumferentially surround the capillaries at the top of the hole and the lower portion which supports the capillaries at the bottom ends of the capillaries.

In various embodiments, the base further comprises: a top surface having a plurality of funnel-shaped holes formed therein, and a support structure, located beneath the top surface, which provides rigidity to the top surface.

In various embodiments, the base further comprises: an upper portion including a top surface having the plurality of funnel-shaped holes arranged in the grid-like pattern which support vertically oriented capillaries by providing circumferential support to the capillaries; and a lower portion providing the inside bottom of the container and providing the positions vertically aligned with the tops of the holes of the top surface, the lower portion supporting the capillaries at the bottom ends of the capillaries.

In another aspect, the present invention provides an injection molded polymeric capillary container which holds ninety-six capillaries in a vertical position. The container comprises: a polymeric base which holds the capillaries in a vertical position by a top surface with ninety-six holes which circumferentially surround the- capillaries, with the capillaries extending over one-half inch above the top surface, and a bottom which supports the bottom ends of the capillaries in ninety-six predetermined positions which are vertically aligned with the holes of the top surface; and a polymeric cover which removably fits over the top of the base and covers the capillaries that are located in the base.

In various embodiments, the polymer is electrically conductive to retard static buildup. In some embodiments, the polymeric base comprises two portions: an upper portion having a structurally reinforced top surface containing ninety-six funnel shaped holes for receiving capillaries; and a lower portion which press-fits together with the upper portion, the lower portion having ninety-six centering supports which support the ends of capillaries in vertical alignment with the funnel shaped holes and are tapered to locate the ends of the capillaries at the ninety-six positions.

In various embodiments, the polymeric base further includes a shoulder which defines the position of the cover when the cover is fit over the top of the base, the defined position providing a space between the cover and the base into which capillaries loaded into the container can extend, the capillaries extending over half of the distance between the top of the base and the cover.

In various embodiments, the present invention provides a capillary container comprising: a removable cover which can be secured on top of the container, the cover providing clearance for the upper portions of a plurality of capillaries which are loaded into the container; and a base having a plurality of holes arranged in a grid-like pattern which hold capillaries in an upright vertical position, the holes holding the capillaries in a vertical orientation by circumferentially surrounding the capillaries, the base having a lower portion with positions aligned vertically with the tops of the holes which support the capillaries at the bottom ends of the capillaries, wherein said container holds 96 capillaries with about 4.5 mm center-to-center spacing.

In various embodiments, the present invention provides an injection molded polymeric capillary container which holds ninety-six capillaries in a vertical position. The container comprises: a polymeric base which holds the capillaries in a vertical position by a top surface with ninety-six holes which circumferentially surround the- capillaries, with the capillaries extending over one-half inch above the top surface, and a bottom which supports the bottom ends of the capillaries in ninety-six predetermined positions which are vertically aligned with the holes of the top surface; and a polymeric cover which removably fits over the top of the base and covers the capillaries that are located in the base, wherein said container holds capillaries with about 4.5 mm center-to-center spacing.

In various embodiments, the present invention provides a method for detecting protein phosphorylation in a sample without using phosphorylation-specific antibodies. Some embodiments of the method comprises: resolving a sample comprising a phosphorylated protein in a fluid path with isoelectric focus electrophoresis; immobilizing the protein in said fluid path; contacting the protein with detection agent which binds to or interacts with said phosphorylated protein; and detecting said phosphorylated protein by detecting said detection agent.

### BRIEF DESCRIPTION OF THE DRAWINGS

FIG. 1A depicts one embodiment of a system according to the present invention in which a standard is first added to a sample to be analyzed. FIG. 1B is a schematic diagram illustrating one embodiment of the method of the present invention carried out in a capillary immunoassay according to embodiments of the invention.

FIG. 2 is a schematic drawing depicts an exemplary process of conducting an immunoassay according to some embodiments of the invention.

FIG. 3A is a side plan view illustrating a general layout of an automated system according to some embodiments of the present invention. FIG. 3B is a top plan view illustrating an exemplary layout of a resource tray of the automated system.

FIGS. 4A and 4B are control system diagrams depicting an exemplary assay flow using the automated system according to some embodiments of the present invention.

FIG. 5 is a system block diagram of an exemplary embodiment illustrating automation of the system of the present invention.

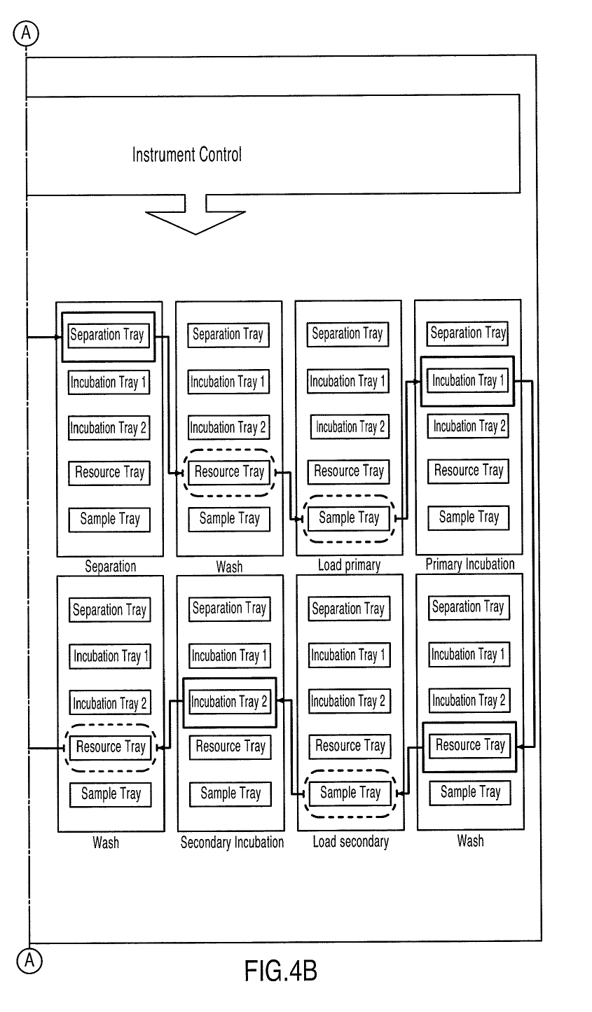

FIG. 6 is a cross-sectional plan view of an embodiment of the automated system.

FIG. 7 is a cross-sectional plan view of a portion of the automated system showing the temperature control mechanism for the incubation and separation stations according to some embodiments of the present invention.

FIG. 8A is top plan view of a UV lamp according to one exemplary embodiment of the present invention. FIG. 8B is a cutaway perspective view of the UV lamp.

FIG. 9 is a perspective assembly drawing of the cover and upper and lower sections of the base of a capillary storage and dispensing container constructed in accordance with some embodiments of the present invention.

FIGS. 10a-10d are plan and cross-sectional views of the cover of a capillary storage and dispensing container of FIG. 9.

FIG. 11 is a perspective view of the base of a capillary storage and dispensing container according to some embodiments of the present invention.

FIG. 12 is a perspective view, looking upward at the underside of the base of FIG. 11.

FIGS. 13a-13g are plan and cross-sectional views of the upper section of the base of a capillary storage and dispensing container according to some embodiments of the present invention.

FIGS. 14a-14e are plan and cross-sectional views of the lower section of a base of a capillary storage and dispensing container according to some embodiments of the present invention which fits together with the upper section of FIG. 13a-13g.

FIG. 15 is a cutaway perspective view of a capillary storage and dispensing container of the present invention loaded with capillaries according to some embodiments of the present invention.

### DETAILED DESCRIPTION OF THE INVENTION

It is to be understood that both the foregoing general description and the following detailed description are exemplary and explanatory only and are not restrictive of the methods and devices described herein. In this application, the use of the singular includes the plural unless specifically stated otherwise. Also, the use of “or” means “and/or” unless state otherwise. Similarly, “comprise,” “comprises,” “comprising,” “include,” “includes,” “including” “haves” and “having” are not intended to be limiting.

The present invention provides compositions, methods and systems for separation, immobilization, and detection of various target analytes. In some embodiments, the system of the present invention is configured to provide the functionality of both pipettes and fluid paths for analysis in a single system. This enables analysis of very small volume samples, and among other advantages improves overall fluid consumption as well as simplifying automation and improving robustness.

### I. Methods of Separating And Detecting the Target Analyte

In one aspect, the present invention provides a method for separating a target analyte in a test sample. In some embodiments, a method is provided comprising the steps of: adding the test sample to a fluid path, separating the target analyte using electrophoresis, immobilizing the target analytes, and detecting the presence of the target analyte. In some embodiments, the test sample may be already contained in a fluid path, such as a capillary, so the step of adding a test sample is omitted.

In some embodiments, the method further comprises adding an internal standard to the sample prior to adding the sample to the fluid path and/or detecting the internal standard.

### Target Analyte

In one aspect, the present invention provides methods and compositions useful in the detection of target analytes. By “target analyte” or “analyte” or grammatical equivalents herein is meant any molecule or compound to be detected and that can bind to a binding species, such as a detection molecule or reagent, as described herein. Suitable analytes include, but not limited to, small chemical molecules such as environmental or clinical chemical or pollutant or biomolecule, including, but not limited to, pesticides, insecticides, toxins, therapeutic and abused drugs, hormones, antibiotics, antibodies, organic materials, etc. Suitable biomolecules include, but are not limited to, proteins (including enzymes, immunoglobulins and glycoproteins), nucleic acids (DNA and RNA), lipids, lectins, carbohydrates, hormones, whole cells (including procaryotic (such as pathogenic bacteria) and eucaryotic cells, including mammalian tumor cells), viruses, spores, etc. Particularly preferred analytes are proteins including enzymes, drugs, cells, antibodies, antigens, cellular membrane antigens and receptors (neural, hormonal, nutrient, and cell surface receptors) or their ligands.

By “proteins” or grammatical equivalents herein is meant proteins, oligopeptides and peptides, and analogs, including proteins containing non-naturally occurring amino acids and amino acid analogs, and peptidomimetic structures.

Sample preparation may be performed as follows. By “sample” herein is meant a composition that contains the analyte or analytes to be detected. The sample can be heterogeneous, containing a variety of components, i.e. different proteins. Alternatively, the sample can be homogenous, containing one component. The sample can be naturally occurring, a biological material, or man-made material. The material can be in a native or denatured form. The sample can be a single cell or a plurality of cells, a blood sample, a tissue sample, a skin sample, a urine sample, a water sample, or a soil sample. In some embodiments, the sample comprises the contents of a single cell, or the contents of a plurality of cells. The sample can be from a living organism, such as a eukaryote, prokaryote, mammal, human, yeast, or bacterium, or the sample can be from a virus.

In some embodiments, the sample can be one or more stem cells. A stem cell is any cell that has the ability to divide for indefinite periods of time and to give rise to specialized cells. Suitable examples include embryonic stem cells, such as human embryonic stem cells (hES), and non-embryonic stems cells, such as mesenchymal, hematopoietic, induced pluripotent stem cells (iPS cells), or adult stem cells (MSC).

As will be appreciated by those skilled in the art, virtually any processing may be performed on the sample prior to detecting the analyte. For example, the sample can be subjected to a lysing step, denaturation step, heating step, purification step, precipitation step, immunoprecipitation step, column chromatography step, centrifugation, etc. In some embodiments, the separation of the sample and immobilization may be performed on native substrates, the analyte of interest, i.e. a protein, or may also undergo denaturation to expose their internal hydrophobic groups for immobilizing in the fluid path.

The analyte to be detected can be any analyte selected by the user. The analyte can comprise any organic or inorganic molecule capable of being detected. Non-limiting examples of analytes that can be detected include proteins, oligopeptides and peptides, derivatives and analogs, including proteins containing non-naturally occurring amino acids and amino acid analogs. Other example of analytes that can be detected include carbohydrates, polysaccharides, glycoproteins, viruses, metabolites, cofactors, nucleotides, polynucleotides, transition state analogs, inhibitors, drugs, nutrients, electrolytes, hormones, growth factors and other biomolecules as well as non-biomolecules, as well as fragments and combinations of all the forgoing.

### Standards

In one aspect, the present invention is directed to the use of one or more internal standards for measuring or quantifying analytes. Generally, a known quantity of standard is added to a sample comprising one or more analytes, and both the standard and the sample are separated by capillary electrophoresis. Then the standard and the analyte(s) are detected with one or more detection molecules or reagents, such as with an antibody against the analyte or a labeling moiety attached to the standard. The signal of the standard and the signal of the analyte(s) are compared to measure the analyte(s) in the sample.

In one aspect, the present invention provides an internal standard. By “standard” or “internal standard” herein is meant a well characterized substance of known amount that is added to an unknown analyte for comparative purposes. Internal standards serve to calibrate the separation with respect to isoelectric point, or for an alternative separation mode, molecular weight. Internal standards for IEF are well know in the art, for example see, Shimura, K., Kamiya, K., Matsumoto, H., and K. Kasai 10 (2002) Fluorescence-Labeled Peptide pI Markers for Capillary Isoelectric Focusing, Analytical Chemistry v74 at 1046-1053, and U.S. Pat. No. 5,866,683. Standards to be detected by fluorescence could be illuminated either before or after chemiluminescence, but generally not at the same time as chemiluminescence.

In some embodiments, an internal standard is a purified form of the analyte itself, although it is generally preferred that the standard be distinguishable from the analyte in some way. This can be performed by a variety of ways, such as by making trivial changes that do not alter the relevant properties of the standard. Preferably an internal standard is different from the analyte but behaves in a way similar to the analyte, enabling relevant comparative measurements.

In some embodiments the standard of the invention is comprised of a purified and well characterized form of the analyte. Any method of obtaining a pure form of the analyte is compatible with the invention, including but not limited to purification from nature, purification from organisms grown in the laboratory, by chemical synthesis and the like.

In some embodiments the standard of the invention has been altered in some way that distinguishes it from the unknown when detected in the detection step or system. The distinguishing characteristic can be any change that is compatible with the invention, including but not limited to dye labeling, radiolabeling, or modifying the mobility of the standard during the electrophoretic separation so that it is separated from the analyte. For example, a standard can contain a modification of the analyte that changes the charge, or mass, or length of the standard relative to the analyte of interest. Modifications include but are not limited to a deletion, fusion, or any chemical modification.

In some implementations, the analyte and standards are detected by fluorescence. The analyte and standards can each be labeled with fluorescent dyes that are each detectable at discrete emission wavelengths, such that the analyte and standards are independently detectable.

In some embodiments, standards that are suitable for use in the present invention are described in U.S. Pat. App. Pub. No. 20070062813, which is incorporated herein by reference in its entirety.

In exemplary embodiments, electrophoresis standards are generally comprised of: one or more moieties capable of affecting electrophoretic mobility, capable of detection, and capable of immobilizing the standard. In some embodiments, the electrophoresis standard comprises one or more moieties capable of immobilizing the standard by covalently linking the standard to a substrate. Typically, the one or more moieties includes one or more functional groups configured to exhibit or perform the desired functionality.

In one embodiment, the invention provides an electrophoresis standard comprising: a compound comprised of one or more moieties, at least one of said moieties being comprised of one or more reactive moieties, wherein the reactive moieties, when activated, attach the electrophoresis standard to a substrate.

In some embodiments, the invention provides electrophoresis standards having a general formula of:

LM−MM−RM

where LM is one or more label moieties, MM is one or more mobility moieties and RM is one or more reactive moieties, and as described in detail below.

In other embodiments, the electrophoresis standard further comprises one or more mobility moieties. The mobility moiety can comprise any entity capable of affecting electrophoretic mobility of the standard. The electrophoretic mobility of the entire compositions can be largely dominated by the properties of the mobility moiety. Properties of the mobility moiety that can affect electrophoretic mobility include, but are not limited to, molecular weight, charge to mass ratio, pI, and hydrophobicity.

The mobility moiety can be any organic and/or inorganic molecule, synthetic or naturally-occurring monomer, oligomer or polymer and any combinations thereof. In some embodiments, the mobility moiety can be an amino acid, peptide, oligopeptide, protein, nucleotide, polynucleotide, carbohydrate, polysaccharide, lipid, ampholyte, dye, heterocycles, and any combinations thereof.

In some embodiments, the electrophoresis standard comprises from 1 to 1000 or more amino acids. The amino acids can be L-amino acid, D-amino acid, an amino acid analog and any combinations thereof. In some embodiments, the standard comprises a modified amino acid. The modified amino acid can make the standard resistant to proteolysis.

The electrophoresis standards comprise one or more label moieties (LM) capable of detection. The label moiety, as will be appreciated by those in the art, can encompass a wide variety of possible labels. In general, labels include, optical dyes, including colored or fluorescent dyes; chemiluminescent labels, phosphorescent labels, enzymatic labels such as alkaline phosphatase and horseradish peroxidase, bioluminescent labels, isotopic labels, which may be radioactive or heavy isotopes, mass labels and particles such as colloids, magnetic particles, etc.

In some embodiments, the label moiety can be a single isomer dye. In some embodiments, the label moiety comprises a fluorescent dye. The fluorescent dye can comprise any entity that provides a fluorescent signal and that can be used in accordance with the methods and devices described herein. Typically, the fluorescent dye comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, non-limiting examples include xanthenes, rhodamines, fluoresceins, cyanines, phthalocyanines, squaraines, bodipy dyes, coumarins, oxazines, and carbopyronines. In some embodiments, the fluorescent dye is 5-carboxytetramethylrhodamine (5-TAMRA).

The electrophoresis standards comprise one or more reactive moieties (RM). In some embodiments the reactive moieties are capable of immobilizing the standard. Immobilization may be accomplished by a variety of methods. For example, in some embodiments, the electrophoresis standards comprise one or more reactive moieties capable of covalently linking the standards to a substrate. In this example, activation of the reactive moieties covalently links the standard to substrate relative to the analyte of interest so that standard and analyte can be compared. In some embodiments, the standard comprises two or more reactive moieties. In embodiments employing two or more reactive moieties, each reactive moiety can be the same, or some or all of the reactive moieties may differ. Having two or more reactive moieties can increase immobilization of the standard by increasing the number of bonds between the standard and a substrate. For example, it can be desirable to increase the immobilization efficiency of the standard in a capillary IEF.

A wide variety of reactive moieties suitable for covalently linking two molecules together are well-known; however such reactive moieties have not been synthesized in combination with other moieties to form the compositions of the present invention. The actual choice of reactive moieties will depend upon a variety of factors, and will be apparent to those of skill in the art based on the teaching of the present invention herein. For example, the reactive moiety can bind to carbon-hydrogen (C—H) bonds of proteins. Since many separation media also contain components with C—H bonds, compounds that react with sulfhydryl (S—H) groups may be advantageous in that S—H groups are found uniquely on proteins relative to most separation media components. Compounds that react with amine or carboxyl groups may also be advantageous due to the prevalence of such groups on proteins.

Suitable reactive moieties (RM) include, but are not limited to, photoreactive groups, chemical reactive groups, and thermoreactive groups.

When the reactive moiety is comprised of one or more photoreactive groups, in some embodiments the photoreactive groups are comprised of one or more latent photoreactive groups that upon activation by an external energy source, forms a covalent bond with other molecules. A list of suitable latent photoreactive groups are described in U.S. Pat. Nos. 5,002,582 and 6,254,634, the disclosures of which are incorporated herein by reference. These photoreactive groups generate active species such as free radicals and particularly nitrenes, carbenes, and excited states of ketones upon absorption of electromagnetic energy. Additionally, photoreactive groups can be chosen that are responsive to various portions of the electromagnetic spectrum, such as those responsive to ultraviolet, infrared and visible portions of the spectrum. For example, upon exposure to a light source, the photoreactive group can be activated to form a covalent bond with an adjacent molecule.

Suitable photoreactive groups include, but are not limited to, aryl ketones, azides, diazos, diazirines, and quinones.

In some embodiments, the photoreactive group comprises aryl ketones, such as benzophenone, acetophenone, anthraquinone, anthrone, and anthrone-like heterocycles or their substituted derivatives. Benzophenone is a preferred photoreactive moiety, since it is capable of photochemical excitation with the initial formation of an excited singlet state that undergoes intersystem crossing to the triplet state. The excited triplet state can insert into carbon-hydrogen bonds by abstraction of a hydrogen atom to create a radical pair. The subsequent collapse of the radical pair leads to formation of a new carbon-carbon bond. If a reactive bond (e.g., carbon-hydrogen) is not available for bonding, the ultraviolet light-induced excitation of the benzophenone group is reversible and the molecule returns to ground state energy level upon removal of the energy source.

In other embodiments, photoreactive groups are comprised of azides, such as arylazides such as phenyl azide, 4-fluoro-3-nitrophenyl azide, acyl azides such as benzoyl azide and p-methylbenzoyl azide, azido formates such as ethyl azidoformate, phenyl azidoformate, sulfonyl azides such as benzenesulfonyl azide, and phosphoryl azides such as diphenyl phosphoryl azide and diethyl phosphoryl azide.

Photoreactive groups may also be comprised of diazo compounds and includes diazoalkanes such as diazomethane and diphenyldiazomethane, diazoketones such as diazoacetophenone and 1-trifluoromethyl-1-diazo-2-pentanone, diazoacetates such as t-butyl diazoacetate and phenyl diazoacetate, and beta-keto-alpha-diazoacetates such as t-butyl alpha diazoacetoacetate.

In further embodiments, photoreactive groups are comprised of diazirines such as 3-trifluoromethyl-3-phenyldiazirine, and photoreactive group comprises ketenes such diphenylketene.

In yet further embodiments, photoreactive groups are comprised of N-((2-pyridyldithio)ethyl)-4-azidosalicylamide, 4-azido-2,3,5,6-tetrafluorobenzoic acid, 4-azido-2,3,5,6-tetrafluorobenzyl amine, benzophenone-4-maleimide, benzophenone-4-isothiocyanate, or 4-benzoylbenzoic acid.

As described above, in embodiments employing two or more reactive moieties, each reactive moiety can be the same, or some or all of the reactive moieties may differ. For example, electrophoresis standards of the invention can comprise a photoreactive group (RM1) and a chemically reactive group (RM2). In some embodiments, electrophoresis standards are comprised of different photoreactive groups, non limiting examples include, two photoreactive groups of benzophenone and 4-azido-2,3,5,6-tetrafluorobenzoic acid (ATFB).

In addition to the use of photoactivatable chemistry described above, chemical or thermal activation may also be employed where free radical are formed due to dissociation of peroxides or azo compounds attached to the surfaces of the fluid paths, or to the particles deposited onto the surface of the fluid path. These thermoactivatible functional groups may also be utilized in solution or as a part of the polymeric compositions added to solutions.

In some embodiments, the reactive moiety (RM) comprises a functional group that is configured to attach the standard to a substrate by forming a covalent linkage with a complementary group present on a substrate. Pairs of complementary groups capable of forming covalent linkages are known in the art and can be selected given the teaching of the present invention. In some embodiments, the substrate is formed of a material that comprises a nucleophilic group and the reactive group comprises an electrophilic group. In other embodiments, the reactive group comprises a nucleophilic group and the substrate is comprised of a material that comprises an electrophilic group. Complementary nucleophilic and electrophilic groups, or precursors thereof that can be suitably activated, useful for forming covalent linkages stable in assay conditions can be used. Examples of suitable complementary nucleophilic and electrophilic groups, as well as the resultant linkages formed there from, are described in U.S. Pat. No. 6,348,596, which is incorporated herein by reference.

Electrophoresis standards of the present invention may be synthesized to exhibit a broad range of characteristics and mobilities. In some embodiments, electrophoresis standards exhibit an isoelectric point in the range of about pH 2 to about pH 12. In some embodiments, electrophoresis standards have a molecular weight in the range of about 20 Da to about 800 kDa.

Methods of making electrophoresis standards are provided in U.S. Pat. App. Pub. No. 20070062813, which is incorporated by reference.

### Fluid Paths And Capillaries

In general, the assay of the present invention is carried out in an assay system described herein using a capillary. However, the assay is not limited to a capillary-based system and can be carried in a variety of fluid path as described herein.

The fluid path can comprise any structure that allows liquid or dissolved molecules to flow. Thus, the fluid path can comprise any structure known in the art, so long as it is compatible with the methods and devices described herein. In some embodiments, the fluid path is a bore or channel through which a liquid or dissolved molecule can flow. In some embodiments, the fluid path is passage in a permeable material in which liquids or dissolved molecules can flow.

The fluid path comprises any material that allows the detection of the analyte within the fluid path. The fluid path comprises any convenient material, such as glass, plastic, silicon, fused silica, gel, or the like. In some embodiments, the method employs a plurality of fluid paths. A plurality of fluid paths enables multiple samples to be analyzed simultaneously.

The fluid path can vary as to dimensions, width, depth and cross-section, as well as shape, being rounded, trapezoidal, rectangular, etc., for example. The fluid path can be straight, rounded, serpentine, or the like. As described below, the length of the fluid path depends in part on factors such as sample size and the extent of sample separation required to resolve the analyte or analytes of interest.

In some embodiments, the fluid path comprises a tube with a bore, such as a capillary. In some embodiments, the method employs a plurality of capillaries. Suitable sizes include, but are not limited to, capillaries having internal diameters of about 10 to about 1000 μm, although more typically capillaries having internal diameters of about 25 to about 400 μm can be utilized. Smaller diameter capillaries use relatively low sample loads while the use of relatively large bore capillaries allows relatively high sample loads and can result in improved signal detection.

The capillaries can have varying lengths. Suitable lengths include, but are not limited to, capillaries of about 2 to 20 cm in length, although somewhat shorter and longer capillaries can be used. In some embodiments, the capillary is about 3, 4, 5, or 6 cms in length. Longer capillaries typically result in better separations and improved resolution of complex mixtures. Longer capillaries can be of particular use in resolving low abundance analytes.

Generally, the capillaries are composed of fused silica, although plastic capillaries and PYREX (i.e., amorphous glass) can be utilized. As noted above, the capillaries do not need to have a round or tubular shape, other shapes, so long as it is compatible with the methods and devices described herein can also be utilized.

In some embodiments, the fluid path can be a channel. In some embodiments, the method employs a plurality of channels. In some embodiments, the fluid path can be a channel in a microfluidic device. Microfluidics employs channels in a substrate to perform a wide variety of operations. The microfluidic devices can comprise one or a plurality of channels contoured into a surface of a substrate. The microfluidic device can be obtained from a solid inert substrate, and in some embodiments in the form of a chip. The dimensions of the microfluidic device are not critical, but in some embodiments the dimensions are in the order of about 100 μtm to about 5 mm thick and approximately about 1 centimeters to about 20 centimeters on a side. Suitable sizes include, but are not limited to, channels having a depth of about 5 μm to about 200 μm, although more typically having a depth of about 20 μm to about 100 μm can be utilized. Smaller channels, such as micro or nanochannels can also be used, so long as it is compatible with the methods and devices described herein.

In some embodiments, the fluid path comprises a gel. In some embodiments, the gel is capable of separating the components of the sample based on molecular weight. A wide variety of such gels are known in the art, a non-limiting example includes a polyacrylamide gel.

The methods generally comprise resolving one or more analytes, contained in a sample, in the fluid path. Methods of separating a mixture into two or more components are well know to those of ordinary skill in the art, and may include, but are not limited to, various kinds of electrophoresis. As used herein, electrophoresis refers to the movement of suspended or dissolved molecules through a fluid or gel under the action of an electromotive force applied to electrodes in contact with a fluid.

### Separation

In another aspect, the method provided by the instant invention comprises separating the target analyte, such as by using electrophoresis. The methods generally comprise resolving one or more analytes, contained in a sample, in the fluid path. Methods of separating a mixture into two or more components are well know to those of ordinary skill in the art, and may include, but are not limited to, various kinds of electrophoresis.

As will be appreciated by those in the art, virtually any method of loading the sample in the fluid path may be performed. For example, the sample can be loaded into one end of the fluid path. In some embodiments, the sample is loaded into one end of the fluid path by hydrodynamic flow. For example, in embodiments wherein the fluid path is a capillary, the sample can be loaded into one end of the capillary by hydrodynamic flow, such that the capillary is used as a micropipette. In some embodiments, the sample can be loaded into the fluid path by electrophoresis, for example, when the fluid path is gel filled and therefore more resistant to hydrodynamic flow.

Embodiments of the invention include separation of the analytes by any physical characteristic, including but not limited to their size, pI, or charge to mass ratio, hydrophobicity, etc.

In some embodiments the sample and standard are subjected to an electrophoretic separation. By “electrophoresis” herein is meant the movement of suspended or dissolved molecules through a fluid or gel under the action of an electromotive force applied to electrodes in contact with a fluid. In some embodiments the fluid or gel contains one or more buffers. In some embodiments the buffers are carrier ampholytes suitable for isoelectric focusing. In some embodiments, the fluid path comprises a gel. In some embodiments, the gel is capable of separating the components of the sample based on size, length, or molecular weight. A wide variety of such gels are known in the art, a non-limiting example includes a polyacrylamide gel.

In some embodiments, resolving one or more analytes comprises isoelectric focusing (IEF) of a sample. In an electric field, a molecule will migrate towards the pole (cathode or anode) that carries a charge opposite to the net charge carried by the molecule. This net charge depends in part on the pH of the medium in which the molecule is migrating. One common electrophoretic procedure is to establish solutions having different pH values at each end of an electric field, with a gradient range of pH in between. At a certain pH, the isoelectric point of a molecule is obtained and the molecule carries no net charge. As the molecule crosses the pH gradient, it reaches a spot where its net charge is zero (i.e., its isoelectric point) and it is thereafter immobile in the electric field. Thus, this electrophoresis procedure separates molecules according to their different isoelectric points.

In some embodiments, for example when resolving is performed by isoelectric focusing, an ampholyte reagent can be loaded into the fluid path. An ampholyte reagent is a mixture of molecules having a range of different isoelectric points. Typical ampholyte reagents are Pharmalyte™ and Ampholine™ available from GE Healthcare. Ampholytes can be supplied at either end of the fluid path, or both, by pumping, capillary action, gravity flow, electroendosmotic pumping, or electrophoresis, or by gravity siphon that can extend continuously through the fluid path.

In some embodiments, resolving one or more analytes comprises electrophoresis of a sample in a polymeric gel. Electrophoresis in a polymeric gel, such as a polyacrylamide gel or an agarose gel separates molecules on the basis of the molecule's size. A polymeric gel provides a porous passageway through which the molecules can travel. Polymeric gels permit the separation of molecules by molecular size because larger molecules will travel more slowly through the gel than smaller molecules.

In some embodiments, resolving one or more analytes comprises micellar electrokinetic chromatography (MEKC) of a sample. In micellar electrokinetic chromatography, ionic surfactants are added to the sample to form micelles. Micelles have a structure in which the hydrophobic moieties of the surfactant are in the interior and the charged moieties are on the exterior. The separation of analyte molecules is based on the interaction of these solutes with the micelles. The stronger the interaction, the longer the solutes migrate with the micelle. The selectivity of MEKC can be controlled by the choice of surfactant and also by the addition of modifiers to the sample. Micellar electrokinetic chromatography allows the separation of neutral molecules as well as charged molecules.

### Immobilization of the Analyte And the Standard

In another aspect, the methods provided in the present invention comprise immobilizing one or more resolved analytes and the standard in the fluid path. By “immobilizing” herein is meant to substantially reduce or eliminate the motion of molecules in the fluid path. The analytes can be immobilized by any methods compatible to the present invention, including but not limited to chemical, photochemical, and heat treatment. The immobilization can be via covalent bonds or non-covalent means such as by hydrophobic or ionic interaction. In some embodiments, the resolved analytes of the sample are immobilized in the fluid path after the analytes have been separated by isoelectric focusing.

In some embodiments, the fluid path comprises one or more reactive moieties. A reactive moiety can be used to covalently immobilize the resolved analyte or analytes in the fluid path. The reactive moiety can comprise any reactive group that is capable of forming a covalent linkage with a corresponding reactive group of individual molecules of the sample. Thus, the reactive moiety can comprise any reactive group known in the art, so long as it is compatible with the methods and devices described herein. In some embodiments, the reactive moiety comprises a reactive group that is capable of forming a covalent linkage with a corresponding reactive group of an analyte of interest. In embodiments employing two or more reactive moieties, each reactive moiety can be the same, or some or all of the reactive moieties may differ.

The reactive moiety can be attached directly, or indirectly to the fluid path. In some embodiments, the reactive moiety can be supplied in solution or suspension, and may form bridges between the wall of the fluid path and the molecules in the sample upon activation. The reactive moiety can line the fluid path or, in another embodiment, may be present on a linear or cross-linked polymer in the fluid path. The polymer may or may not be linked to the wall of the fluid path before and/or after activation.

A wide variety of reactive moieties suitable for covalently linking two molecules together are well-known. The actual choice of reactive moieties will depend upon a variety of factors, and will be apparent to those of skill in the art. For example, the reactive moiety can bind to carbon-hydrogen (C—H) bonds of proteins. Since many separation media also contain components with C—H bonds, chemistries that react with sulfhydryl (S—H) groups may be advantageous in that S—H groups are found uniquely on proteins relative to most separation media components. Chemistries that react with amine or carboxyl groups may also be advantageous due to the prevalence of such groups on proteins.

Suitable reactive moieties include, but are not limited to, photoreactive groups, chemical reactive groups, and thermoreactive groups.

Photoimmobilization in the fluid path can be accomplished by the activation of one or more photoreactive groups as described here that related to the standards.

In some embodiments, the reactive moiety comprises a functional group that can be converted to a functionality that adheres to an analyte via hydrophobic interactions, ionic interactions, hydrogen bonding etc. In some embodiments, such reactive moieties are activated with the UV light, laser, temperature, or any other source of energy in order to immobilize the analytes onto the surfaces of the fluid paths and/or onto the surfaces of particles attached to the surfaces of fluid paths. In some embodiments, the surfaces of the fluid paths are functionalized with thermally responsive polymers that enable changes in hydrophobicity of the surfaces upon changing the temperature. In some embodiments, the analytes are immobilize on such surfaces by increasing hydrophobicity of a temperature responding polymer when a certain temperature is reached within the fluid path.

### Detection Agents

In some embodiments, the methods comprise contacting one or more analytes with one or more detection agents. A detection agent is capable of binding to or interacting with the analyte to be detected. Contacting the detection agent with the analyte or analytes of interest can be by any method known in the art, so long as it is compatible with the methods and devices described herein. Examples for conveying detection agents through the fluid path include, but are not limited to, hydrodymic flow, electroendosmotic flow, or electrophoresis.

The detection agents can comprise any organic or inorganic molecule capable of binding to interact with the analyte to be detected. Non-limiting examples of detection agents include proteins, peptides, antibodies, enzyme substrates, transition state analogs, cofactors, nucleotides, polynucleotides, aptamers, lectins, small molecules, ligands, inhibitors, drugs, and other biomolecules as well as non-biomolecules capable of binding the analyte to be detected.

In some embodiments, the detection agents comprise one or more label moiety(ies). In embodiments employing two or more label moieties, each label moiety can be the same, or some, or all, of the label moieties may differ.

In some embodiments, the label moiety comprises a chemiluminescent label. The chemiluminescent label can comprise any entity that provides a light signal and that can be used in accordance with the methods and devices described herein. A wide variety of such chemiluminescent labels are known in the art. See, e.g., U.S. Pat. Nos. 6,689,576, 6,395,503, 6,087,188, 6,287,767, 6,165,800, and 6,126,870 the disclosures of which are incorporated herein by reference. Suitable labels include enzymes capable of reacting with a chemiluminescent substrate in such a way that photon emission by chemiluminescence is induced. Such enzymes induce chemiluminescence in other molecules through enzymatic activity. Such enzymes may include peroxidase, beta-galactosidase, phosphatase, or others for which a chemiluminescent substrate is available. In some embodiments, the chemiluminescent label can be selected from any of a variety of classes of luminol label, an isoluminol label, etc. In some embodiments, the detection agents comprise chemiluminescent labeled antibodies.

In some embodiments, the detection agents comprise chemiluminescent substrates. Depending on their charge, the chemiluminescent substrates can be supplied from either end of the fluid path, once the analyte is immobilized in the fluid path. Uncharged substrates can be supplied from either end of the fluid path by hydrodynamic flow or electroendosmotic flow, for example. Chemiluminescent substrates are well known in the art, such as Galacton substrate available from Applied Biosystems of Foster City, Calif. or SuperSignal West Femto Maximum Sensitivity substrate available from Pierce Biotechnology, Inc. of Rockford, Ill. or other suitable substrates.

Likewise, the label moiety can comprise a bioluminescent compound. Bioluminescence is a type of chemiluminescence found in biological systems in which a catalytic protein increases the efficiency of the chemiluminescent reaction. The presence of a bioluminescent compound is determined by detecting the presence of luminescence. Suitable bioluminescent compounds include, but are not limited to luciferin, luciferase and aequorin.

In some embodiments, the label moiety comprises a fluorescent dye. The fluorescent dye can comprise any entity that provides a fluorescent signal and that can be used in accordance with the methods and devices described herein. Typically, the fluorescent dye comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, non-limiting examples include xanthenes, rhodamines, fluoresceins, cyanines, phthalocyanines, squaraines, bodipy dyes, coumarins, oxazines, and carbopyronines. In some embodiments, for example, where detection agents contain fluorophores, such as fluorescent dyes, their fluorescence is detected by exciting them with an appropriate light source, and monitoring their fluorescence by a detector sensitive to there characteristic fluorescence emission wavelength. In some embodiments, the detection agents comprise fluorescent dye labeled antibodies.

In embodiments, using two or more different detection agents, which bind to or interact with different analytes, different types of analytes can be detected simultaneously. In some embodiments, two or more different detection agents, which bind to or interact with the one analyte, can be detected simultaneously. In various embodiments, using two or more different detection agents, one detection agent, for example a first antibody, can bind to or interact with one or more analytes to form a detection agent-analyte complex, and second detection agent, for example a second antibody, can be used to bind to or interact with the detection agent-analyte complex.

In some embodiments, two different detection agents, for example antibodies for both phospho- and non-phospho-forms of analyte of interest can enable detection of both forms of the analyte of interest. In some embodiments, a single specific detection agent, for example an antibody, can allow detection and analysis of both phosphorylated and non-phosphorylated forms of a analyte, as these can be resolved in the fluid path. In some embodiments, multiple detection agents can be used with multiple substrates to provide color-multiplexing. For example, the different chemiluminescent substrates used would be selected such that they emit photons of differing color. Selective detection of different colors, as accomplished by using a diffraction grating, prism, series of colored filters, or other means allow determination of which color photons are being emitted at any position along the fluid path, and therefore determination of which detection agents are present at each emitting location. In some embodiments, different chemiluminescent reagents can be supplied sequentially, allowing different bound detection agents to be detected sequentially.

In some embodiments a detection molecule or reagent is an antibody capable of binding to or interacting with the standard and the analyte to be detected. Contacting the detection agent with the analyte or analytes of interest can be by any method known in the art, so long as it is compatible with the methods and devices described herein. Examples for conveying detection agents through the capillary include, but are not limited to, hydrodynamic flow, electroendosmotic flow, or electrophoresis.

Antibodies used for detection of the standard and the analyte may be labeled by any means compatible with the invention such as but not limited to fluorescent dyes, optical dyes, chemiluminescent reagents, radioactivity, particles, magnetic particles, etc. In some embodiments, the detection agents comprise one or more label moiety(ies). In embodiments employing two or more label moieties, each label moiety can be the same, or some, or all, of the label moieties may differ.

### Detection

Detection of the analytes can be performed by any methods compatible with the invention, including but not limited to chemiluminescence, staining, autoradiography, fluorescence, and the like.

Analyte detection includes detecting of the presence or absence, measurement, and/or characterization of an analyte. Typically, an analyte is detected by detecting a signal from a label and includes, but is not limited, to detecting isotopic labels, immune labels, optical dyes, enzymes, particles and combinations thereof such as chemiluminescent labeled antibodies and fluorescent labeled antibodies.

Detecting the analyte can be by any method known in the art, so long as it is compatible with the methods and devices described herein. Analyte detection can be performed by monitoring a signal using conventional methods and instruments, non-limiting examples include, a photodetector, an array of photodetectors, a charged coupled device (CCD) array, etc. For example, a signal can be a continuously monitored, in real time, to allow the user to rapidly determine whether an analyte is present in the sample, and optionally, the amount or activity of the analyte. In some embodiments, the signal can be measured from at least two different time points. In some embodiments, the signal can be monitored continuously or at several selected time points. Alternatively, the signal can be measured in an end-point embodiment in which a signal is measured after a certain amount of time, and the signal is compared against a control signal (sample without analyte), threshold signal, or standard curve.

A signal can be a monitored, in real time, to allow the user to rapidly determine whether an analyte is present in the sample, and optionally, the amount or activity of the analyte. In some embodiments, the signal can be measured from at least two different time points. In some embodiments, the signal can be monitored continuously or at several selected time points. Alternatively, the signal can be measured in an end-point embodiment in which a signal is measured after a certain amount of time, and the signal is compared against a control signal (sample without analyte), threshold signal, or standard curve.

Typically, detecting the analyte comprises imaging the fluid path. In some embodiments, the entire length of the fluid path can be imaged. Alternatively, a distinct part or portion of the fluid path can be imaged. The amount of the signal generated is not critical and can vary over a broad range. The only requirement is that the signal be measurable by the detection system being used. In some embodiments, a signal can be at least 2-fold greater than the background. In some embodiments, a signal between 2 to 10-fold greater than the background can be generated. In some embodiments, a signal can be 10-fold greater than the background.

The amount of the signal generated is not critical and can vary over a broad range. The only requirement is that the signal be measurable by the detection system being used. In some embodiments, a signal can be at least 2-fold greater than the background. In some embodiments, a signal between 2 to 10-fold greater than the background can be generated. In some embodiments, a signal can be 10-fold greater than the background.

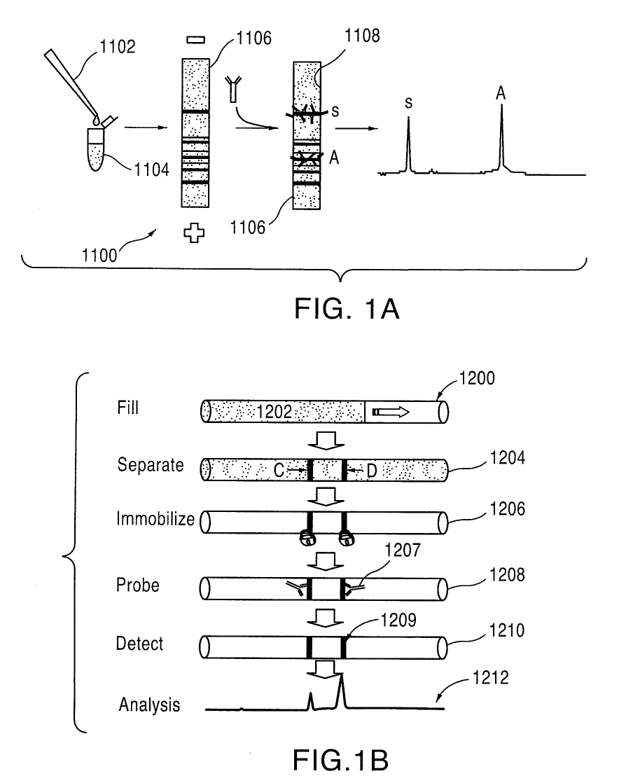

FIG. 1A is a schematic illustration depicting one embodiment of a system 1100 according to the present invention in which a standard 1102 is first added to a sample 1104 to be analyzed. The sample 1104 is then subjected to an electrophoretic separation in a capillary 1106 in which the standard (S) and the analyte (A) are separated from each other. Then a potion or the entire sample is immobilized to a wall 1108 of the capillary 1106. The sample and the standard are then probed simultaneously with a single detection molecule, reagent or agent that binds to both the standard (S) and the analyte (A) of interest. The detection reagent is then used to create a signal that can be detected and graphed as signal vs. length of the capillary. The signal from the standard (S) can then be used to interpret the signal from the analyte (A).

FIG. 1B is a schematic diagram illustrating one embodiment of the method of the present invention carried out in a capillary immunoassay system of the present invention. In this embodiment, capillaries were prepared as described in U.S. Pat. App. Pub. No. 20060029978, which is herein incorporated by reference in its entirety. Briefly, the capillaries 1200 are loaded with a separation medium and the sample 1202. The contents are resolved by capillary electrophoresis shown at 1204. Resolved proteins are then immobilized to the capillary wall as shown at 1206. The immobilized proteins are then probed with an analyte-specific antibody, and a HRP-conjugated secondary antibody in a manner similar to a western blot as shown at 1208. Because the protein-antibody complexes are immobilized in the capillary, detection molecules or reagents can be flowed through the capillary as shown at 1210. Light generated from where the antibodies bound is imaged onto a CCD camera. Data is extracted and plotted as signal intensity vs. capillary length with the analyte showing up as a peak as shown at 1212. Fluorescent standards are used to align the data from capillary to capillary. Also within the scope of the present invention are a variety of variations on the methods described above.

### Bright/Dim Internal Standards

In another aspect, the present invention provides bright/dim internal standards for the detection of an analyte or molecule of interest. Applications include but are not limited to proteins separated by electrophoresis and immobilized within capillaries. Embodiment of the system provided herein is used to carry out a variety of assays, particularly immunoassays carried out in a capillary.

In general, during the standard immunoassay process, a portion of the internal standard will be lost due to the various wash processes. Thus, it is generally desirable to load a sufficient amount of internal standard in the sample at the beginning of the assay so that enough signal can be generated by the internal standard that remains in the capillary after the immunoassay to provide coordination to calibrate the curve and analyze the size or pI of the analyte. However, and without being bound by any particular theory, it is believed that a large amount of internal standard may interfere with the capture of the analyte if the standard and the analyte are located in the same position. Thus, it is desirable to have a standard that does not locate with the analyte during and/or at the end of the electrophoresis. However, such standard may not produce a reliable calibration curve for the detection of the analyte. The present invention provides an internal standard comprised of two standards, a dim standard that locates close or to at the same position as the analyte and a bright standard that does not locate at the same position as the analyte. The dim standard provides an accurate calibration curve and the bright standard provides coordinate (an anchor point) for the dim standard.

In various embodiments, the internal standard of the present invention is a bright/dim standard that comprises two standards, a bright standard and dim standard.

“Bright standard” or “registration standard” as used herein is a standard that has characteristics (such as molecular weight or pI) that differs from that of the analyte such as after electrophoresis the location of registration standard and the analyte are located apart from each other in the capillary. Thus, the fluorescence emitted from the bright standard and the analyte will not overlap and interfere with each other.

“Dim standard” or “additional standard” as used herein is a standard that has characteristics (such as molecular weight or IE) that are similar to that of the analyte such as after electrophoresis the location of the registration standard and the analyte are located close to each other in the capillary.

Generally, the bright standard produces a fluorescence that is brighter than the fluorescence emitted by the dim standard after the internal standard and the analyte have been separated and immobilized. The difference of the brightness between the bright standard and dim standard may due to the difference in the nature of emission or due to the difference in the amounts of the two standards contained in the internal standard. For example, a large quantity of bright standard and a small quantity of dim standard can be mixed to form a standard that can produce a “bright” signal from the bright standard and a “dim” signal from the dim standard. Thus, a “bright” signal due to the bright standard and a “dim” signal due to the dim signal are detected after the separation step by electrophoresis. After the various wash steps are conducted for the detection of the analyte, certain amounts of both the bright standard and the dim standard are lost such that a weak signal or no signal can be detected from the dim standard. However, the signal due to the bright standard is still detectable, although the signal may be weaker than before the various washing steps, which provides the coordinates for locating both the dim standard and the analyte.

The brightness of the standard can be measured in various way. The fluorescent signal may be initiated with broad area excitation or with one or more focused scanned beams and the resulting signal may be detected using an array detector such as a CCD or, for a scanned beam, a point detector such as a photomultiplier tube or an avalanche photodiode. In some embodiments, the brightness is measured by the signal generated by the standard as captured by a CCD detector.

The bright and dim standards can be comprised of a variety of standards as described herein as long as they migrate independently during electrophoresis such that the bright standard is separate from the analytes and the dim standard preferably migrates closely to the analyte.

In some embodiments, the bright standard and the dim standard comprise the same fluorophore, and thus are excited and emit at the same wavelength.

In some embodiments, the bright standard and the dim standard may comprise different fluorophores, and are excited at the same wavelength but emit at different wavelengths; or alternatively are excited at different wavelengths but emit at the same wavelength. In yet another alternative, both are excited and emit at different wavelengths.

In some embodiments, the difference in the brightness between the bright standard and the dim standard is due to the difference in the amount of the bright standard relative to the dim standard contained in the internal standard. Generally, the amount of the bright standard is about 5-20 times or more than the dim standard. In one embodiment, the amount of the bright standard is about 10 times of the dim standard.

In some embodiments, the difference in the brightness between the bright standard and the dim standard is due to the difference in the fluorophore, such as different emission intensity and/or difference in the number of fluorescence moiety per molecule of the standard.

The selection of the bright standard and the dim standard depends on the analyte to be detected as well as the type of electrophoresis used for the separation. In general, the preferred relation between the bright standards, dim standards, and the target analytes depends on the range of pI or molecular weight arrayed across the capillary. The preferred separation of the bright standard from the analyte is sufficient to yield a resolution of greater than 1 but less than 4 where the resolution is equal to the distance between the peaks of the analyte and standard divided by twice the sum of the peak widths of the standard and the analyte. The dim standard is as close as practical to the analyte in either pI or molecular weight.

In various embodiments, the internal standard comprises one bright standard and one dim standard. In some embodiments, the internal standard comprises one bright standard and a plurality of dim standards. This is especially suitable when more than one analytes are detected. In some embodiments, the internal standard comprises a plurality of bright standards and plurality of dim standards.

In one aspect, the present invention provides a method of measuring one or more analytes in a sample. The method comprises adding an internal standard to the sample, the internal standard comprising a first standard and a second standard. Then the sample is loaded into a microfluidic device. The internal standard and the analyte are separated by electrophoresis. This is followed by the immobilizing the internal standard and the analyte. A first image comprising the image of the first standard and the second standard is captured, followed by the detection the analyte with an antibody to produce a second image. A third image comprising the image of the first standard is captured. Finally, the analyte is measured by comparing the first image, the second image and the third image.

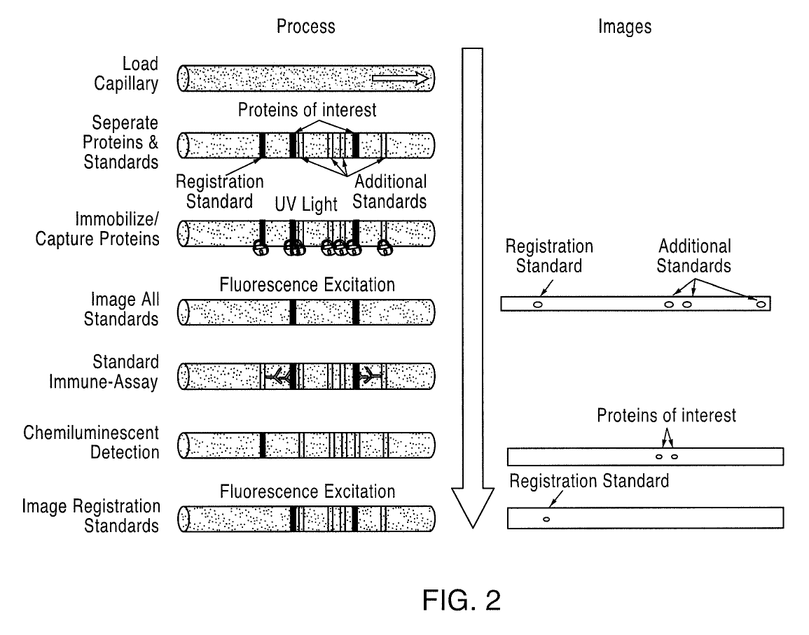

FIG. 2 depicts one embodiment of an assay according to the present invention in which a standard is first added to a sample to be analyzed. The sample contains proteins of interest. The standard comprises a registration standard (the bright standard) and an additional standard (the dim standard), both are labeled with a label moiety comprising a fluorescent dye, such as those described herein. The sample is then subjected to an electrophoretic separation in a capillary in which the standard and the analyte (a protein of interest) are separated from each other. Then a portion or the entire sample is immobilized to a wall of the capillary, such as by illuminating of UV light. This is followed by excitation of the fluorescent dye by a light and a first image which captures all standards is taken, such as using a digital camera. The sample is then probed with a single detection molecule, reagent or agent that binds to the analyte of interest, such as a chemiluminescence labeled antibodies against the analyte. A second image that captures all the chemiluminescence signals that corresponding to the analyte is taken. This is followed by excitation of the fluorescent dye again by a light and a third image which captures all standards is taken. The three images are measured and graphically represented as signal vs. length of the capillary. The signal from the standard can then be used to interpret the signal from the analyte.

### Kits

In another aspect of the present invention kits for performing the methods described herein, and for analyte detection systems are provided. In one embodiment, the kit comprises materials for making the electrophoresis standards described herein. Additionally, one or more mobility moieties, one or more reactive moieties, one or more label moieties are provided. In some embodiments, the kit comprises one or more electrophoresis standards as described herein. In some embodiments, the kit further comprises electrophoresis standard comprising a peptide, one or more fluorescent dyes and one or more photoreactive groups. Additional materials can include, but are not limited, fluid paths, such as capillaries and microfluidic devices. In addition, buffers, polymeric or polymerizable materials, blocking solutions, and washing solutions can be provided. In some embodiments, the kit can further comprise reagents for the activation of a reactive moiety. These other components can be provided separately from each other, or mixed together in dry or liquid form.

In an exemplary embodiments, the present invention provides a kit for measuring at least one analyte in a sample using electrophoresis. The kit comprises an internal standard described herein. In various embodiments, the amount of the bright standard is about 5-20 times or more than the amount of dim standard. In one embodiment, the amount of the bright standard is about 10 times the amount of the dim standard.

In various exemplary embodiments, the kit comprises a cell lysis reagent. The cell lysis agent can be any suitable cell lysis agent known in the art, such as the Pierce Cell Lysis Reagents supplied by Thermo Fisher Scientific Inc.

### Phosphorylation Assay

In another aspect, the present invention provide methods for the detection of phosphorylation of a protein.

Protein phosphorylation is one of the most important general mechanisms of cellular regulation. Protein phosphorylation commonly occurs on three major amino acids, tyrosine, serine or threonine, and changes in the phosphorylation state of these amino acids within proteins can regulate many aspects of cellular metabolism, regulation, growth and differentiation. Changes in the phosphorylation state of proteins, mediated through phosphorylation by kinases, or dephosphorylation by phosphatases, is a common mechanism through which cell surface signaling pathways transmit and integrate information into the nucleus. Given their key role in cellular regulation, it is not surprising that defects in protein kinases and phosphatases have been implicated in many disease states and conditions. For example, the over-expression of cellular tyrosine kinases such as the EGF or PDGF receptors, or the mutation of tyrosine kinases to produce constitutively active forms (oncogenes) occurs in many cancer cells. Drucker et al. (1996) Nature Medicine 2: 561-56. Protein tyrosine kinases are also implicated in inflammatory signals. Defective Thr/Ser kinase genes have been demonstrated to be implicated in several diseases such as myotonic dystrophy as well as cancer, and Alzheimer's disease (Sanpei et al. (1995) Biochem. Biophys. Res. Commun. 212: 341-6; Sperber et al (1995) Neurosci. Lett. 197: 149-153; Grammas et al (1995) Neurobiology of Aging 16: 563-569; Govoni et al. (1996) Ann. N.Y. Acad. Sci. 777: 332-337).

The involvement of protein kinases and phosphatases in disease states makes them attractive targets for the therapeutic intervention of drugs, and in fact many clinically useful drugs act on protein kinases or phosphatases. Examples include cyclosporin A which is a potent immunosuppressant that binds to cyclophilin. This complex binds to the Ca/calmodulin-dependent protein phosphatase type 2B (calcineurin) inhibiting its activity, and hence the activation of T-cells. (Sigal and Dumont (1992), Schreiber and Crabtree (1992)). Inhibitors of protein kinase C are in clinical trails as therapeutic agents for the treatment of cancer. (Clin. Cancer Res. (1995) 1:113-122) as are inhibitors of cyclin dependent kinase. (J. Mol. Med. (1995) 73:(10):509-14.)

The number of known kinases and phosphatases are growing rapidly as the influence of genomic programs to identify the molecular basis for diseases have increased in size and scope. These studies are likely to implicate many more kinase and phosphatase genes in the development and propagation of diseases in the future, thereby making them attractive targets for drug discovery. However, current methods of measuring protein phosphorylation have many disadvantages which prevents or limits the ability to rapidly screen using miniaturized automated formats of many thousands of compounds. This is because current methods rely on the incorporation and measurement of ³²P into the protein substrates of interest. In whole cells this necessitates the use of high levels of radioactivity to efficiently label the cellular ATP pool and to ensure that the target protein is efficiently labeled with radioactivity. After incubation with test drugs, the cells must be lysed and the protein of interest purified to determine its relative degree of phosphorylation. This method requires high numbers of cells, long preincubation times, careful manipulation and washing steps (to avoid artifactual phosphorylation or dephosphorylation), as well as a method of purification of the target protein. Furthermore, final radioactive incorporation into target proteins is usually very low, giving the assay poor sensitivity.

Alternative assay methods, for example based on phosphorylation-specific antibodies using ELISA-type approaches, involve the difficulty of producing antibodies that distinguish between phosphorylated and non-phosphorylated proteins, and the requirement for cell lysis, multiple incubation and washing stages which are time consuming, complex to automate and potentially susceptible to artifacts.

The present invention provides a method for detecting protein phosphorylation in a sample without using phosphorylation-specific antibodies. Some embodiments of the method comprises: resolving a sample comprising a phosphorylated protein in a fluid path with isoelectric focus electrophoresis; immobilizing the protein in said fluid path; contacting the protein with detection agent which binds to or interacts with said phosphorylated protein; and detecting said phosphorylated protein by detecting said detection agent.

In some embodiments, the sample further comprises non-phosphorylated form of the protein. Preferably, the detection agent is an agent that binds to or interacts with both the phosphorylated and non-phosphorylated forms of the protein, such as an antibody, which can be either monoclonal or polyclonal. In various embodiments, the method comprises quantification and/or comparison of the phosphorylated and non-phosphorylated forms of the protein. Such method can be used to assay the kinases or phosphatases that phosphorylate and dephosphorylate the protein.

In various embodiments, the sample contains both the phosphorylated and non-phosphorylated forms of the protein.

In some embodiments, the sample comprises an internal standard of known pI and the detecting step comprises detecting the internal standard and measuring the pI of the proteins. In some embodiments, the internal standard comprises the bright and dim standards provided herein.

In another aspect, the present invention provides a method of measuring phosphorylated and non-phosphorylated forms of a protein in a sample. Some embodiments of the method comprise: adding an internal standard to a sample comprising phosphorylated and non-phosphorylated forms of a protein, the internal standard comprising a bright standard and a dim standard; loading the sample into a microfluidic device; separating the standard and the protein by electrophoresis; immobilizing the internal standard and the protein; capturing a first image comprising the signals generated by the bright standard and the dim standard; detecting the protein with an antibody to produce a second image; capturing a third image comprising the signal generated by the bright standard; and measuring the protein by comparing the first image, the second image and the third image, wherein the antibody recognizes both phosphorylated and non-phosphorylated forms of the protein.

### II. The System

In another aspect, the present invention provides an automated immunoassay system. In some embodiments, the system generally comprises a separation/detection station, wherein the separation/detection station comprises a first or single location for conducting capillary electrophoresis and the detection of a fluorescence during and after the electrophoresis.

The combination of separation and detection in a single station enable the usage of the same light source to induce fluorescence as well as providing the image detector for capturing images during the separation and detection step in real time. Using a single separation/detection chamber also improves the ability to register images containing bright and dim standards taken prior to immunoassay with images taken after immunoassay in which only the bright standards are detectable are described herein.

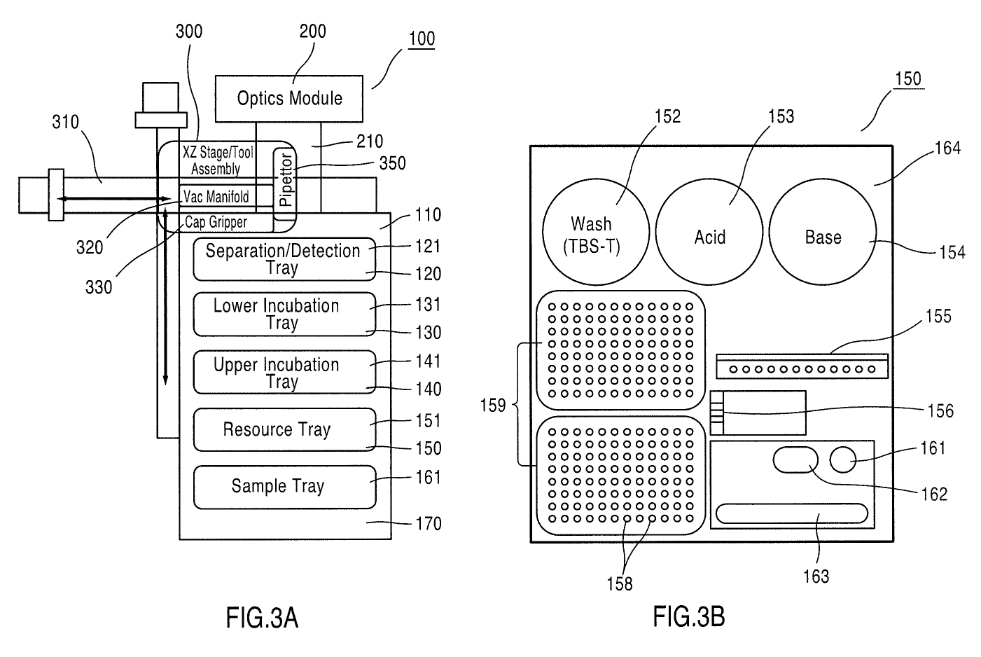

Referring first to FIG. 3A a general layout of an automated system 100 for conducting immunoassay using capillaries is shown. In the illustrative embodiment, the system 100 comprises a chamber 110 that houses a plurality of processing stations: separation/detection station 121, lower incubation station 131, upper incubation station 141, resource station 151 and sample station 161. Each station has a corresponding tray that can move in and out the chamber 110 like drawers: separation/detection tray 120, lower incubation tray 130, upper incubation tray 140, resource tray 150 and sample tray 160. Preferably, the trays are automated to slide into and out from the processing stations in a manner similar to that of a disk tray of a computer optical disk drive. The trays are stacked vertically within chamber 110, which is enclosed within a case 170. Thus, at each processing station, when the trays are in a closed position, the trays and the inner surface of the chamber form individual compartments. The temperature of each compartment is preferably controlled as described in more detail herein. In general, the top chamber is sealed relative to the others when the trays are closed. This is for best optical performance.

Referring again to FIG. 3A, an optics module 200 is mounted on the top of chamber 110 through connection 210. The optics module 200 has a camera that is optically connected to the separation/detection tray 120. When the optics module 200 is installed the area inside separation/detection tray 120 is light tight.

Also shown in FIG. 3A, also amounted on the chamber 110 is an XZ stage 310 that aligns tools in relation to the positions on the chamber trays when extended. The tool components are mounted on a tool assembly 300 and include a vacuum manifold 320, capillary gripper 330 and pipettor 340. Capillary gripper 330 moves capillaries between trays or positions. Suitable grippers are described in WO2006110725, the disclosure of which is herein incorporated by reference in its entirety. Vacuum manifold 320 draws liquids into and through the capillaries. Pipettor 340 transfers, delivers and removes bulk fluids, such as the acid and the base to and from the separation tray/detection tray 120, wash solution to and from the separation tray/detection tray 120, and buffer to and from wash positions in the resource tray 150. The tool assembly 300 is attached to the XZ stage 310 and can move in the X and Z directions.

Sample tray 160 holds a number of microwell plate stations, such as a standard microwell plate contain 96 microwells on a 9 mm center-to-center spacing or 384 microwells on a 4.5 mm center-to-center spacing. Plates with other spacings and numbers of wells may be used and the invention is not intended to be limited to any one specific configuration. Preferably, the microwell plate(s) containing samples are chilled while in these stations. This may be accomplished preferably by thermoelectric cooling or by other means such as refrigeration, recirculating cold fluid or an ice bath coupled to the sample plates. The stations have guides or recesses which precisely define the locations of standard microwell plates when located in the stations. In an exemplary embodiment, the sample tray 160 holds a single standard 384 well plate which is rests on/in a cold plate at about 3° C.

Referring to FIG. 3B, the resource tray 150 has a base 164, on the top of which is mounted a wash bottle 152, an acid bottle 153 and a base bottle 154. The three bottles are each about 30 ml in size and contain TBS buffer, acid and base (e.g., sodium hydroxide), respectively. The resource tray 150 also has two capillary boxes 159 with openings 158 that can hold up to 96 capillaries per box. In a preferred embodiment, the positions have a 4.5 mm center-to-center spacing. Also on the resource tray 150 is a capillary re-grip location 155. In this location, the gripper places the caps in the re-grip location and then picks them back up. This allows the gripper to grab the caps in the middle, which it cannot do when picking them up from the box. Resource tray 150 also has a capillary counter 156 that can count the number of capillaries to confirm that capillaries have not been dropped or lost. Also included in the resource tray 150 is a capillary and pipettor wash block 158, which has a capillary wash reservoir 163 and pipettor wash positions 161 and 162.

Referring again to FIG. 3A, the two incubation trays 130 and 140 each has 48 capillary incubation positions in two rows of 24. In a preferred embodiment, the positions have a 4.5 mm center-to-center spacing.

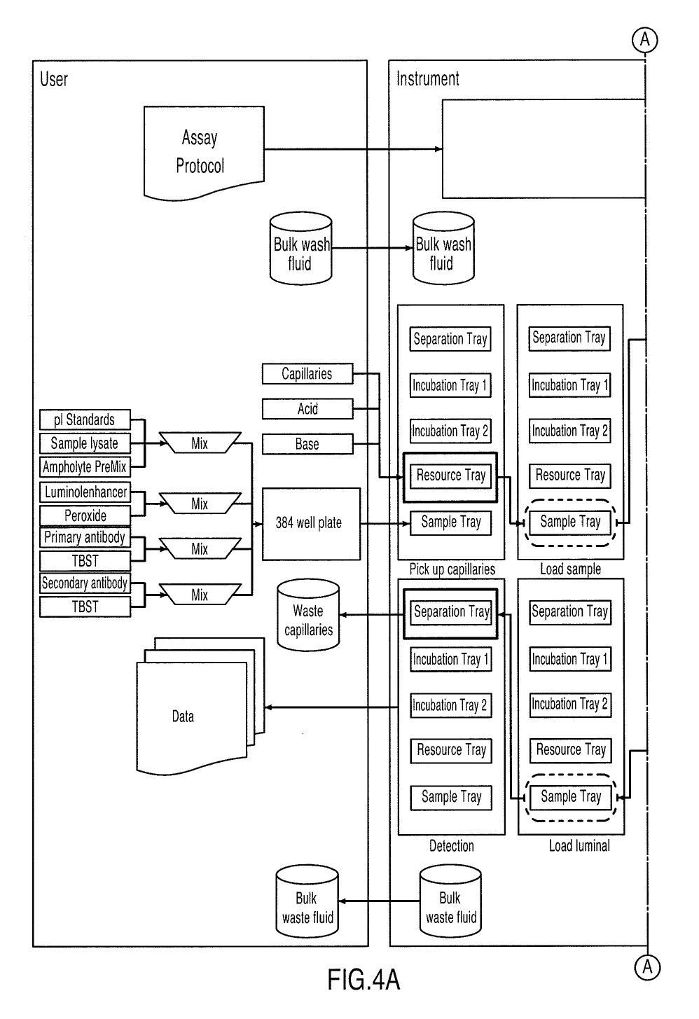

FIGS. 4A and 4B (connected by the line A-A) depict an exemplary assay flow using the system provided herein according to some embodiments of the present invention. The user enters an assay protocol into the instrument control module, which is a computer with memory chips embedded with the programs to run the system and to accept input and provide output. The user also provides bulk wash fluid and other reagents for the assay to be carried out by the system. Specifically, the luminol and the enhancer are mixed with peroxide outside of the system by the user and provided to the system.

A host computer is connected to the system to control the various components of the system as shown in FIGS. 4A and 4B. A power supply assembly provides the power source to the various components of the system.

Referring to FIG. 3A, the optics module houses an imaging optical detector for detecting light emitted from within the capillaries. In various embodiments the optical detector is a cooled charge-coupled device (CCD) array detector. Light from the capillaries is imaged onto the CCD by a lens assembly. The detection station is light-tight when the separation/detection tray 120 is retracted to the interior of the module, enabling the CCD array detector to detect light emitted from a capillary by chemiluminescence or fluorescence.

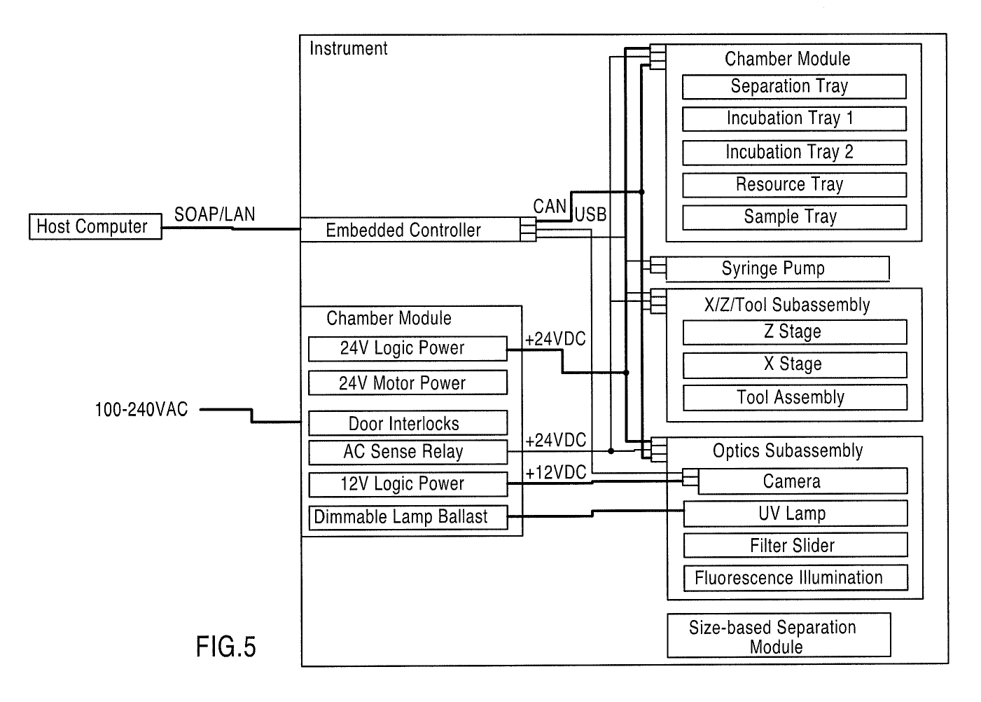

Referring to FIG. 6, in this embodiment, the imaging optical detector is a digital camera 202 that is used for imaging during both the separation and immunoassay detection steps of the assay, as described in more detail herein. The camera has a lens 204 that is optically connected to the separation/detection compartment 121 formed by the separation/detection tray 120 and the inner surface of the chamber 110. The connection is through an opening 206. The camera captures images of the capillaries held by the capillary holder during and/or after the electrophoresis. The camera is used for both luminescent and fluorescent imaging.

To excite fluorescence an array of light emitting diodes (LED) (not shown) is housed inside the separation/detection station 121 and is arranged to uniformly illuminate the capillaries.

The optics module 200 also has a filter slider (not shown) that selects filter to use for imaging. The filters are either clear or TAMRA filters.

Referring again to FIG. 6 the optics module includes UV lamp 102 disposed within the separation/detection station 121. UV lamp 102 that is preferably configured to move to a position over the first location 121 and to illuminates the capillaries held by the capillary holder as described in more details herein.

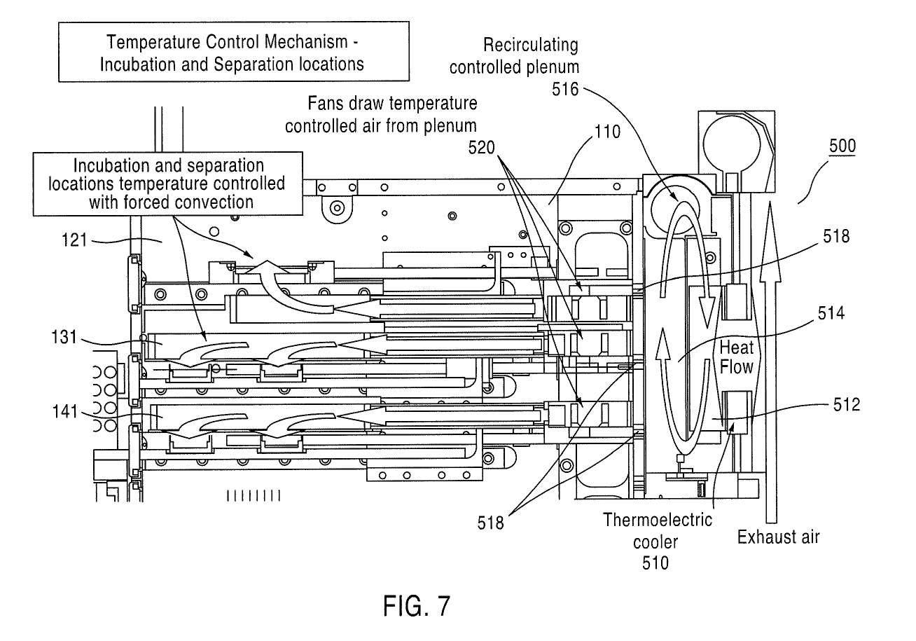

In an exemplary embodiment, the UV lamp 102 is a grid lamp such as the one shown in FIG. 8A. A grid lamp provides uniform area illumination. Preferably, the UV lamp is a low-pressure mercury lamp that generates light at a first wavelength of about 254 nm. In some embodiments, the UV lamp 102 further comprises a phosphor coating to convert the first wavelength of about 254 nm to a second wavelength of about 295 nm. Referring to FIG. 8B, the UV lamp 102 is attached to a lamp cover 103 that ensures the light is spread only in one direction.

Referring to FIG. 6, the separation/detection station 121 has a first location 104 for conducting capillary electrophoresis and the detection of a fluorescence during and after the electrophoresis. The first location 121 has comprises a capillary holder such as those described in WO2006110725, the disclosure of which is herein incorporated by reference in its entirety. The capillary holder may hold a plurality of capillaries so that the CCD array will detect photoemissions from a plurality of capillaries at the same time. In an alternate embodiment a scanning fluorescence detector may be used. In that embodiment excitation light focused by a lens irradiates fluorescent molecules within each capillary. This same (or another) lens collects the resulting fluorescent emission for detection by a photo sensitive device such as a photo-multiplier tube. This focused excitation/collection can be scanned along the length of each capillary individually or in groups. The excitation light is a coherent source such as a laser or an incoherent source such as an arc lamp or light emitting diode array.

In an exemplary embodiments, the capillary holder comprises: first and second fluid reservoirs; a plurality of recesses which retain a plurality of capillaries in position in the holder with the ends of the capillaries located at the first and second reservoirs; and electrodes in contact with each of the first and second fluid reservoirs, wherein fluids in the reservoirs are retained at the respective ends of the capillaries by surface tension.

Following separation of the biological molecules in the fluid paths of the capillaries in accordance with their ionic charge by isoelectric focusing, the separated molecules are immobilized in their focused positions in the capillaries. In an illustrative embodiment, this is accomplished by irradiating the capillaries with ultraviolet light from a UV light source inside the separation/detection station to bind the separated material to either a material within the lumen or to the walls of the capillaries by photoactivated chemistry. Typically, UV light is used at intensity in the range of 1-1000 mW/cm² for a period in the range of 1-200 s. The isoelectrically focused materials are thereafter detected by the optics module 200.

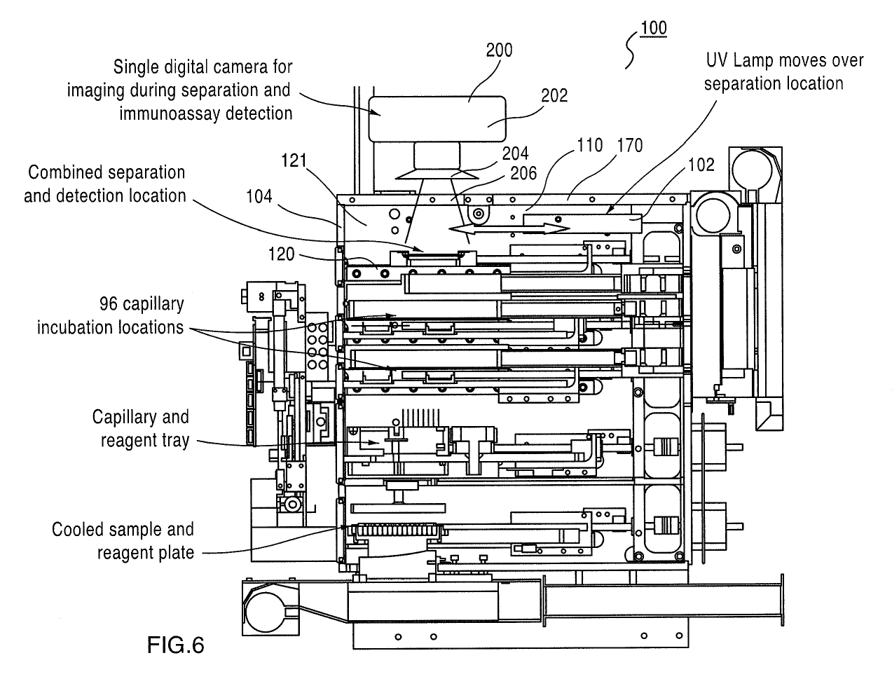

The system of the invention also provides temperature control for the incubation and separation locations. In some embodiments, temperature control is achieved with forced convection. Referring to FIG. 7, a plenum 500 is attached to chamber 110. One side of the plenum 500 is connected to the side of chamber 110 with air passages 518. A thermoelectric cooler 512 is housed at the side of plenum 500 that is opposite to the side attached to the chamber 110. Heat flows through the thermoelectric cooler 512 to or from the plenum 500. Exhaust air flowing pass the outer surface of the thermoelectric cooler transfers heat to or from the outside environment 512. A recalculating control unit 516 controls the circulation of air within the plenum 516, such as flow from passage 512 to passage 514. Within each of the processing stations 121, 131 and 141 located a fan 520 that draws temperature-controlled air from plenum 500.

### III. Container For Transporting And Storing the Capillaries

In another aspect, the present invention provides a container for transporting and storing the capillaries and dispensing them during use of the assay system.

A key to making automation effective without the complexity of machine vision is to know in advance the locations and positions of all of the materials and elements needed to conduct the process, and to program the system computer accordingly to automatically access them.

In the case of the capillaries, a pair of bulk capillary racks are located at specific capillary rack stations on the base of the assay system. The capillaries to be used in the process are initially located in these racks, then moved to a staging rack from which capillaries are selected for use in biological sample processing. The capillary racks hold capillaries upright in rows with a pre-defined center-to-center spacing. The pre-defined spacing permits the capillaries to be removed from the rack by a robotic computer-controlled capillary manipulator which is programmed and controlled to access the capillaries at their known locations.

However, initially loading the capillaries into the racks by hand can be challenging. The capillaries are very small with diameters on the order of 100 μm to 2 mm and lengths ranging from 30 to 100 mm. Handling the capillaries can contaminate them with body oils which can interfere with the optical properties necessary to detect the luminescence emitted from inside the capillaries. The buildup of electrostatic energy can cause both handling problems and attraction of particles which disrupt the use and function of the capillaries. Moreover, in the assay system described herein the capillaries are very closely spaced, with center-to-center spacings ranging from 4.5 mm to 9 mm. The density of capillaries in the capillary racks is also substantial, with a full rack holding 96 to 384 capillaries. The efficiency gained by fully automating the assay processing can be lost to the time required to insert the capillaries into the racks in preparation for system for operation.

Accordingly, it would be desirable for a system user to be able to buy the capillaries from the manufacturer pre-loaded in capillary racks which can be directly used in the capillary rack stations of the assay system, obviating the need to manually handle the capillaries prior to use.

Moreover, it would further be desirable to buy the capillaries pre-coated with the immobilizing coating so that the user does not have to spend time coating the capillaries and enduring the inefficiencies and vagaries associated therewith.

It is further desirable to protect the coated capillaries in containers which keep the capillaries secure from environmental hazards and physical damage prior to use.

It is also desirable to be able to ship and store the capillaries in the same containers, obviating the need to transfer them.

It is generally preferred to have container for capillaries in the standard 96 and 384 configurations. In the 96 configuration, the center-to-center spacings ranging is about 9 mm. In the 384 configuration, the center-to-center spacings ranging is about 4.5 mm. Thus the spacing of the capillaries will be similar to the standard 96 well plates and 384 well plates so that these standard plates can be used to store samples and/regents to be loaded to the capillaries. However, in some embodiments, such standard 9 mm spacing of 96 capillaries is problematic as such spacing requires a much bigger footprint and creates complexity in manipulating the capillaries. The inventors have discovered that contrary to the standard use in the industry, a different configuration as provided by some embodiments of the present invention is advantageous in carrying out the assaying of biological samples. In one embodiment, a narrower spacing is employed which enables advantage for the separation and/or detection steps by promoting easier image capturing. In prior art system, capillaries need to be rearranged between a narrow spacing that is less than 9 mm and a 9 mm spacing. Embodiments of the present invention provides a container with particular spacing between the capillaries that is compatible with the assay system where narrow spacing is used for capillaries handling. Such narrow spacing (about 4.5 mm for 96 capillaries) is different from the standard adopted by the industry, but provide the surprising advantages to enable a faster and simpler process to carry out the assay described in U.S. Pat. App. Pub. Nos.: 20060029978 and 20030032035.

Thus, in another aspect, the present invention provides a container where the capillaries are very closely spaced, with center-to-center spacing ranging from 4.5 mm to 9 mm. The density of capillaries in the capillary racks is also substantial, with a full rack holding 96 to 384 capillaries.

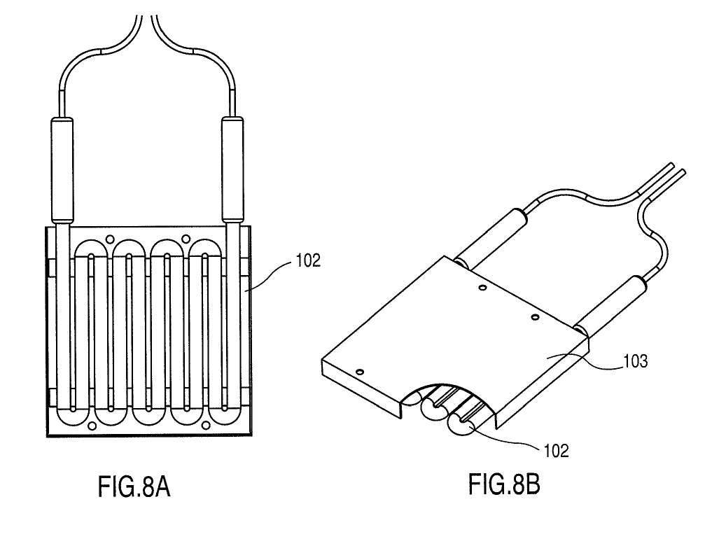

Another exemplary embodiment of the invention is shown in FIGS. 9-15. In this embodiment, the distance between the adjacent holes that hold capillaries is about 4.5 mm, which is about half of the distances of 9 mm in the above described embodiment. This container thus occupies a smaller footprint and enables tighter packing of the capillaries.

Referring first to FIG. 9, the parts of a capillary storage and dispensing container 9210 constructed in accordance with the principles of the present invention are shown in a perspective assembly view. The container can be made of a variety of materials such as metal or plastic. A preferred material is acrylonitrile butadiene styrene (ABS), a thermoplastic copolymer which can advantageously be injection-molded to form the parts of the box. An advantage of ABS is that it combines the strength and rigidity of the acrylonitrile and styrene polymers with the toughness of the polybutadiene rubber. ABS can also be formulated to resist static buildup, which could cause handling or optical problems in the automated assay system. A suitable material is Cycolac® ABS plastic, which is available from GE Plastics of Pittsfield, Mass. ABS can also be formulated with additives to be electrically conductive and thereby reduce static buildup. Suitable ABS polymers with these electrical properties are LNP*Stat-kon* or LNP* Stat-loy*, both available from GE Plastics. Alternatively, the polymeric container can be coated with an anti-static coating.

The container 9210 has a cover 9212 which fits over a base that holds a plurality of capillaries in a vertical, upright position. The base is formed of two sections which press-fit together, an upper section 9230 and a lower section 9260. The bottom portion 9234 of the upper section 9230 is wider than the top portion 9236 so that the cover 9212 will fit over the top portion and cover the capillaries, while the bottom portion fits snugly over and around the lower section 9260 of the base in a secure press-fit. When the top and bottom sections 9230, 9260 are mated together, the capillary holes in the top of the upper section 9230 are in alignment with the capillary receivers of the lower section 9260, which cooperate to hold the capillaries upright with the circumferential holes in the top and the funneled receivers in the bottom. The removable cover 9212 is retained over the top portion 9236 of the base by engagement with two ribs 9232, one of which is molded on either side of the top portion 9236 of the base.

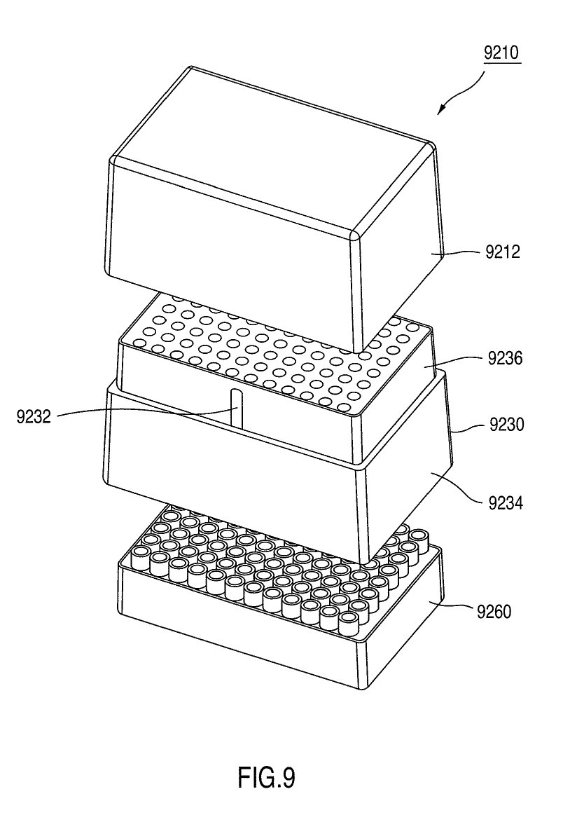

FIGS. 10a-10d show various views of the cover 212. FIG. 10a is a top plan view of the cover 212, which is about 1.6 inches wide, 2.5 inches long, and 1.46 inches high. FIG. 10b is a side plan view of the cover, FIG. 10c is a cross-sectional view taken along cut line 10c of FIG. 10a, and FIG. 10d is a cross-sectional view taken along cut line 10d of FIG. 10a. The cover has a nominal wall thickness 9218 of about 0.08 inches. The sides 9224, 9220 of the cover are slightly inclined outward from the top 9222 to the opening 9216 so that the cover will easily engage the top portion 9236 of the base.

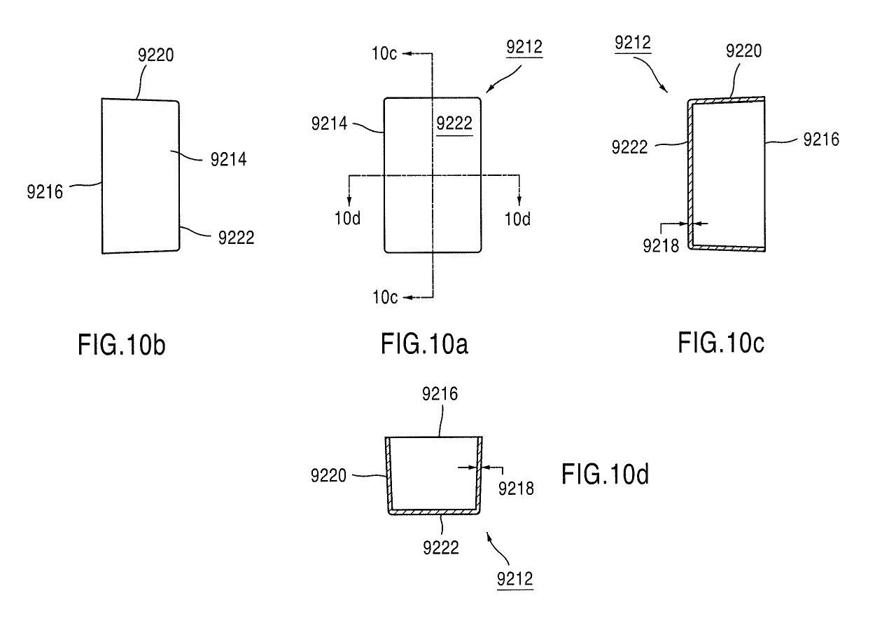

FIG. 11 is a perspective view looking at the top of the assembled base of the container 9210. In this view only the upper section 9230 of the base is visible because in the assembled base the lower section 9260 of the base is fully inside the upper section with the bottom edges of both sections flush with each other and forming the bottom of the base. Holes 9240 which hold the capillaries are formed through the top 9238 of the upper section 9230. Preferably the holes 9240 are in a grid pattern which is familiar to and in common use in the biological assay field so that the container will hold a number of capillaries which is compatible with other assay equipment and devices. In the container shown in the drawings the grid of holes is eight holes wide by twelve holes long and the container will hold ninety-six capillaries when full. This is the same grid pattern as that of the familiar microwell plates used in biological assaying. However, as described herein, while the guiding pattern is similar, the spacing configuration is unique, wherein the container of ninety-six capillaries of the present invention is much smaller than, such as about a quarter of, the size of the ninety-six well capacity of one of the standard microwell plates. Thus, a containers of the present invention is particularly configured as suitable to provide capillaries for the assaying of samples in the ninety-six wells of the plate of a system described herein. Other convenient capillary capacities such as 384 capillaries may also be employed if desired.

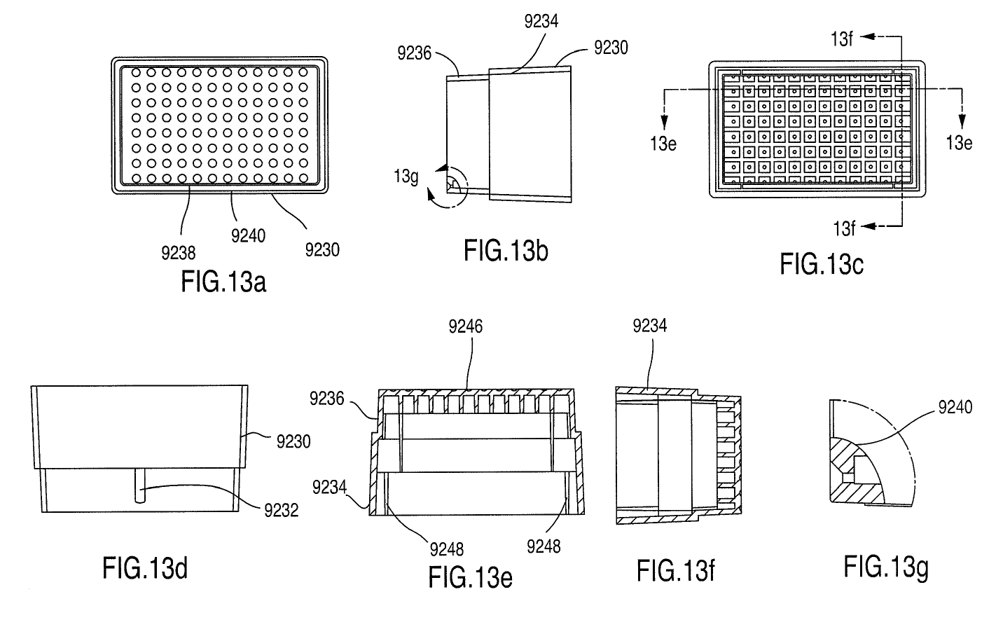

Details of the upper section 9230 are shown in FIGS. 13a-4g. FIG. 13a is a top plan view of the upper section 9230. The section 9230 has a narrower upper portion 9236 which is about one-half inch high, as shown in FIG. 13b. The cover 9212 fits over this upper portion 236 and is retained in place by the two outer ribs 9232. The wider lower portion 9234 is about 0.96 inches high and forms the base of the container. The lower section 9260 of the container fits inside this lower portion 9234 and is retained in contact with ribs 9248. Both portions are slightly tapered on the sides, with the base of the lower portion 9234 measuring about 1.60 by 2.5 inches, which is sized to fit in the profile for a capillary rack on the base of the assay system with which it is to operate. If the footprint of the container is smaller than the size of the capillary rack station of the assay system, an adapter can be provided which fits the system footprint and accommodates the smaller container, in which case the container for the system operably includes the adapter. The top 9238 measures about 1.38 by 2.28 inches as seen in FIGS. 13d and 13f. The ninety-six holes for the capillaries are located in the top surface as shown in FIG. 13a and the holes are evenly spaced on 0.18 inch (4.5 mm) centers in eight rows of twelve holes each as shown in this drawing. At the top the holes are funnel-shaped as best seen in the enlarged cutaway view of FIG. 13g. At the surface of the top 9238 the capillary holes 9240 have a diameter of 0.10 inches which tapers down to a diameter of 0.025 inches in the thickness of the top 9238. The transition from the funnel shape to the constant diameter of the capillary hole 9240 has about a 0.005 inch radius. The nominal diameter of 0.025 inches for the capillary holes is about twice the nominal diameter of a capillary.

To provide rigidity for the top surface 9238 and prevent warping and bending, an egg-crate ribbing 9246 is formed inside the upper portion 9236. The sections of the ribs inside of the periphery are on the same 0.18 inch spacing as the capillary holes 9240. The thickness of the ribbing 9246 is about 0.05 inches as indicated in FIG. 13f. The ribbing 9246 in FIGS. 13e and 13f is about 0.25 inches high in a constructed embodiment, occupying approximately the upper half of the inside of the upper portion 9236.

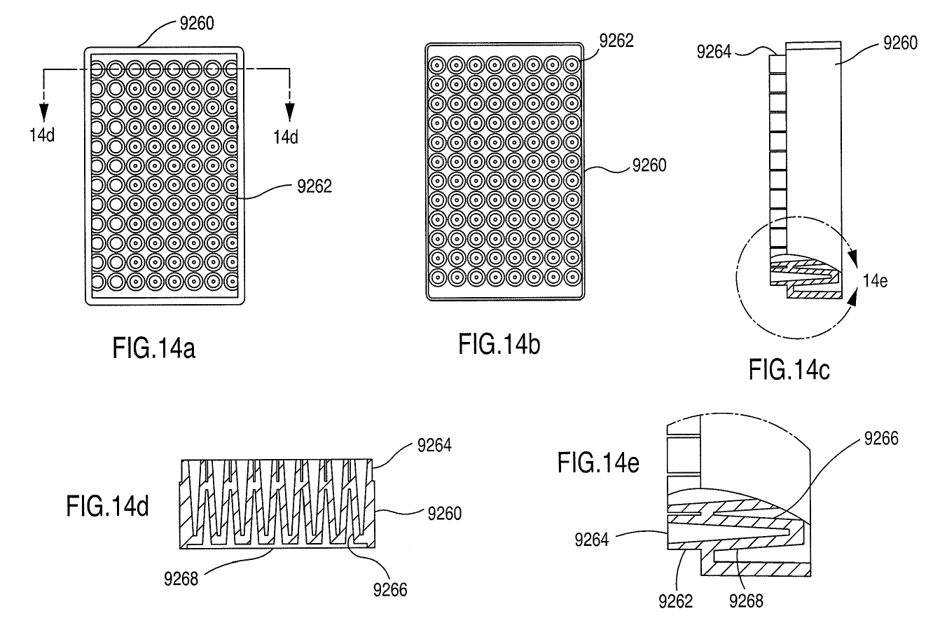

FIGS. 14a-14e are different views of the lower section 9260 of the container which press-fits inside of the upper section 9230. The lower section 9260 supports the capillaries in their upright vertical orientation by supporting the lower ends of the capillaries. Aligned with the holes 9240 of the upper section are ninety-six centering supports 9262 for the lower ends of the capillaries. As indicated in FIG. 14b, these capillary supports 9262 are on the same 0.18 inch center-to-center spacing as the capillary holes 9240. The upper part 9264 of each of the capillary supports 9262 is generally cylindrical with an inner diameter of about 0.1 inches as shown in FIG. 14e, which will easily capture a capillary that is dropped into a hole 9240. The lower part 9266 of the capillary supports 9262 is tapered to a small inner diameter of 0.02 inches at the bottom 268, which is just slightly larger than the diameter of a capillary. Thus, when a capillary is dropped into a hole 9240 of the upper section 9230, the capillary will fall toward the lower section 9260, be captured by the large diameter of the upper part 9264, then be guided by the inner wall of the lower part 9266 to the small bottom area 9268. With the bottom 9268 in alignment with the hole 9240 in the upper section, the capillary will be caused to stay upright in its vertical orientation from which it can be easily and assuredly located and gripped by a capillary gripper of the automated assay system. Thus, the container of the present invention can be used as a capillary rack operable with an automated capillary gripper in an automated assay system.

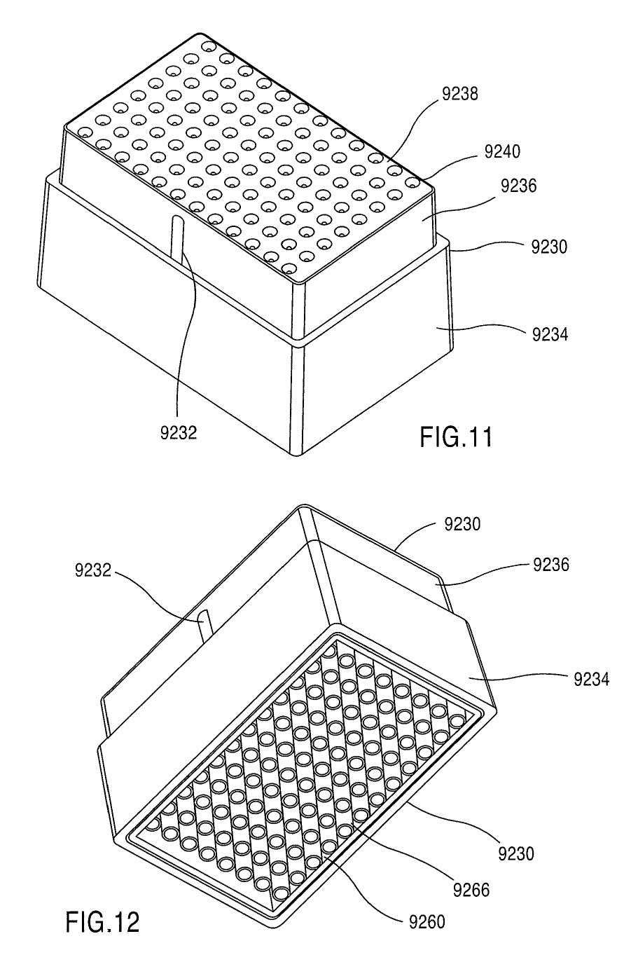

The lower section 9260 measures about 1.6 inches wide by 2.5 inches long as shown in FIGS. 14a and 14b which will snugly press-fit inside the upper section 9230. FIG. 12 is a perspective view looking upward at the container from below after the lower section 9260 has been press-fit inside of the upper section 9230. The outside of the tapered lower parts 9266 of the capillary supports 9262 can be seen inside the lower section 9260 of FIG. 12, just as they can in the views of the lower section 9260 of FIGS. 14a, 14d and 14e.

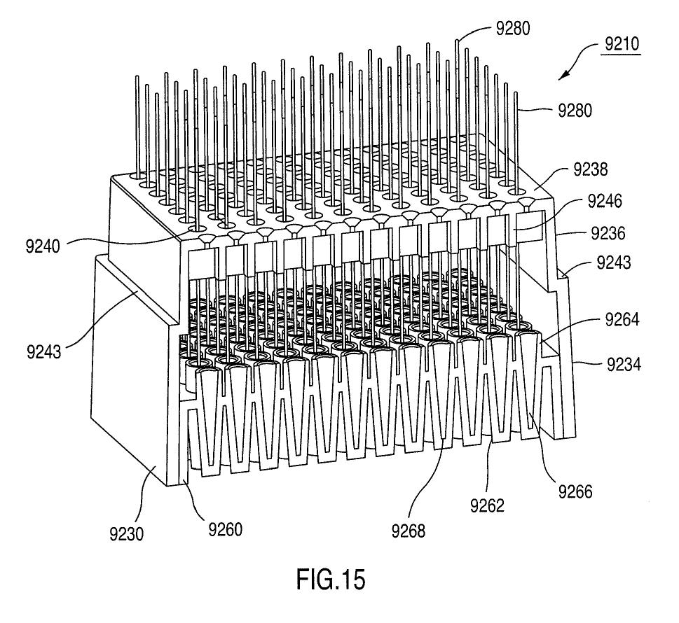

FIG. 15 is a cutaway perspective view of a container 9210 of the present invention with the cover 9212 removed that has been loaded with capillaries 9280. As the drawing shows, when a capillary 280 is inserted into a funnel shaped hole 240 on the top 9238 of the upper portion 9230 of the container, it drops through to the upper part 9264 of an aligned centering support 9262 of the lower section 9260 and the end of the capillary falls to and is supported by the bottom 9268 of a tapered lower part 9266 of the support 9262.

The upper part of each capillary 9280 extends about 0.68 inches above the top surface 9238 of the upper section 9230. A typical capillary is made of glass or a transparent plastic material and is about two inches (50 mm) in length with an outer diameter of about 0.015 inches. When the cover 212 is put in place the cover surrounds the upper portion 9236 of the upper section 9230 of the container down to the shoulder 9243 between the upper and lower portions 9236, 9234 of the section 9230 and provides clearance for the upward extending capillary between the top surface 9238 and the inner surface of the top 9222 of the cover 9212. In a constructed embodiment there is about 0.70 inches of clearance between the top surface 238 and the inner surface of the cover 9212, which prevents the capillaries from coming out of the holes during handling and shipping of a loaded container. When a loaded container has arrived at a user's facility the capillaries 9280 can be stored in the container 9210 until they are to be used. When the capillaries are to be put to use in an automated assay system, the cover 9212 is removed from the container 9210 and the rest of the container, comprising the upper and lower sections 9230 and 9260, loaded with the capillaries 9280, is put on a capillary holder station of the assay system. The capillaries are then ready for automated access and use in an analytical procedure of the assay system.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. An automated assay system, comprising:
a separation/detection station having a location for conducting capillary electrophoresis and detecting a fluorescence at least one of during and after the electrophoresis, the location including a capillary holder having a first fluid reservoir and a second fluid reservoir,
a plurality of recesses configured to retain a plurality of capillaries in a position in the capillary holder with a first end of each capillary from the plurality of capillaries at the first fluid reservoir and a second end of each capillary from the plurality of capillaries at the second fluid reservoir, and
electrodes in contact with each of the first fluid reservoir and the second fluid reservoir, wherein fluids in the first fluid reservoir and the second fluid reservoir are retained at the respective ends of the capillaries by surface tension.

2. The automated assay system of claim 1, wherein said separation/detection station includes a UV lamp configured to move to a position over the location such that the plurality of capillaries are illuminated by UV light.

3. The automated assay system of claim 2, wherein the UV lamp generates light at a wavelength of about 254 nm.

4. The automated assay system of claim 1, further comprising:
an optics module including a camera having a lens optically connected to the separation/detection station and configured to take images at the location.

5. The automated assay system of claim 4, wherein said camera is a digital camera configured to take images both during and after the electrophoresis.

6. A method, comprising:
adding an internal standard to a sample, said internal standard including a bright standard and a dim standard;
loading said sample into a microfluidic device;
separating said internal standard and one or more analytes by electrophoresis;
immobilizing said internal standard and said one or more analytes;
capturing a first image including the signals generated by said bright standard and said dim standard;
detecting said one or more analytes with an antibody to produce a second image;
capturing a third image including the signal generated by said bright standard; and
measuring the one or more analytes by comparing the first image, the second image and the third image.

7. The method of claim 6, wherein said bright standard locates apart from said one or more analytes after the electrophoresis.

8. The method of claim 6, wherein said dim standard locates close to said one or more analytes after the electrophoresis.

9. The method of claim 6, wherein said electrophoresis is isoelectric focusing (IEF).

10. The method of claim 6, wherein said electrophoresis separates the analyte by size.

11. A kit for measuring at least one analyte in a sample using electrophoresis, comprising:
an internal standard including a bright standard and a dim standard,
wherein an amount of said bright standard is greater than an amount of said dim standard such that the bright standard generates a brighter signal than the dim standard when detected, said bright standard locates apart from said analyte after electrophoresis, and said dim standard locates close to said analyte after electrophoresis.

12. The kit of claim 11, wherein said electrophoresis is isoelectric focusing (IEF).

13. A method for detecting protein phosphorylation in a sample, comprising:
resolving a sample including a phosphorylated protein in a fluid path with isoelectric focus electrophoresis;
immobilizing the protein in said fluid path;
contacting the protein with a detection agent; and
detecting said phosphorylated protein by detecting said detection agent, the detection agent configured to at least one of bind or interact with one of a phosphorylated and a non-phosphorylated form of the protein.

14. The method of claim 13, wherein said sample further includes a non-phosphorylated form of the protein.

15. The method of claim 13, wherein said detection agent is an antibody.

16. A method of measuring phosphorylated and non-phosphorylated forms of a protein in a sample, comprising:
adding an internal standard to a sample having phosphorylated and non-phosphorylated forms of a protein, the internal standard including a bright standard and a dim standard;
separating the standard and the protein by electrophoresis;
immobilizing the internal standard and the protein;
capturing a first image including a signal generated by the bright standard and a signal generated by the dim standard;
detecting the protein with an antibody to produce a second image;
capturing a third image including the signal generated by the bright standard; and
measuring the protein by comparing the first image, the second image and the third image, wherein the antibody recognizes both the phosphorylated and the non-phosphorylated forms of the protein.

17. The method of claim 16, wherein said bright standard locates apart from the protein after the electrophoresis.

18. The method of claim 16, wherein said dim standard locates close to the protein after the electrophoresis.

19. The method of claim 16, wherein said electrophoresis is isoelectric focusing (IEF).

20. The method of claim 19, wherein said IEF is performed in a capillary.
